(19) United States
(12) Patent Application Publication
(10) Pub. No.: US 2025/0049853 A1
(43) Pub. Date: Feb. 13, 2025

(54) Treatment of Spinal Cord Injury with T regulatory Cells

(71) Applicant: CREATIVE MEDICAL TECHNOLOGIES, INC., Phoenix, AZ (US)

(72) Inventors: Thomas Ichim, San Diego, CA (US); Courtney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 18/794,184

(22) Filed: Aug. 5, 2024

Related U.S. Application Data
(60) Provisional application No. 63/518,386, filed on Aug. 9, 2023.

(51) Int. Cl. A61K 35/17; A61K 35/28

(52) U.S. Cl. CPC A61K 35/17; A61K 35/28

(57) ABSTRACT

Methods and compositions of matter useful for reducing extent of spinal cord injury and/or reversion of spinal cord injury gliosis are disclosed. T regulatory cells can be administered systemically and/or locally to a patient suffering from spinal cord injury. T regulatory cells can be modulated by engineering either through gene modification or culture conditions in order to augment regenerative/antifibrotic properties. Utilizing pluripotent stem cell derived T regulatory cells for spinal cord injury and chimeric antigen receptor T cells.

### CROSS-REFERENCE TO RELATED APPLICATIONS

The present application claims benefit of U.S. Provisional Patent Application Ser. No. 63/518,386, filed on Aug. 9, 2023, titled TREATMENT OF SPINAL CORD INJURY WITH T REGULATORY CELLS, the contents of which are incorporated herein by reference in its entirety.

### FIELD OF THE INVENTION

The invention pertains to the field of spinal cord injury, more specifically, the invention relates to the field of regenerative approaches for spinal cord injury. More specifically the invention relates to the utilization of T regulatory cells for addressing the issue of suppressing scar formation while concurrently stimulating regeneration.

### BACKGROUND OF THE INVENTION

It is established that spinal cord injury (SCI) is a significant cause of morbidity and mortality. In the United States alone there are estimated to be over 12,000 Americans that are afflicted with SCI annually, and approximately 1.3 million people are living with this condition. Traumatic SCI is mostly found in individuals in their 20s and 30s, resulting in a high-level of permanent disability in young and previously healthy patients. In addition to limitations on limb function, SCI patients suffer from impaired bowel and bladder function, reduced sensation, spasticity, autonomic dysreflexia, thromboses, sexual dysfunction, increased infections, decubitus ulcers and chronic pain, which constitute major causes of morbidity. In terms of mortality, it is accepted that life expectancy of an individual suffering a cervical spinal cord injury at age 20 is 20-25 years lower than that of a similarly aged individual with no SCI (NSCISC Spinal Cord Injury Facts and Figures 2021).

From a pathological perspective the clinical effects of spinal cord injury vary with the site and extent of damage. The neural systems that may be permanently disrupted below the level of the injury not only involve loss of control of limb muscles and the protective roles of temperature and pain sensation, but impact the cardiovascular system, breathing, sweating, bowel control, bladder control, and sexual function These losses lead to a succession of secondary problems, such as pressure sores and urinary tract infections that, until modern medicine, were rapidly fatal. Spinal cord injury often removes those unconscious control mechanisms that maintain the appropriate level of excitability in neural circuitry of the spinal cord. As a result, spinal motoneurons can become spontaneously hyperactive, producing debilitating stiffness and uncontrolled muscle spasms or spasticity. This hyperactivity can also cause sensory systems to produce chronic neurogenic pain and paresthesias, unpleasant sensations including numbness, tingling, aches, and burning. In recent polls of spinal cord injury patients, recovery of ambulatory function was not the highest ranked function that these patients desired to regain, but in many cases, relief from the spontaneous hyperactivity sequelae was paramount.

To date, various potential pharmacological treatments such as steroids, antioxidants, glutamate receptor inhibitors, ion channel inhibitors, gangliosides, antibodies to axon regeneration inhibitors, anti-inflammatory agents, and neurotrophic factors have been tried. However, only methylprednisolone has been used as the therapeutic agent for post-spinal cord injury, and nevertheless, it has many problems, such as unclear therapeutic effects and side effects due to overdose. Therefore, treatment of spinal cord injury using drugs or physiotherapy has reached its limit, and thus many experimental studies focusing on treatment by cell transplantation are in progress. Stem cells have the ability to differentiate throughout the life of an organism for indefinite periods, and treatment methods using them have been studied, but the method has not been established so far.

### SUMMARY OF THE INVENTION

Preferred methods include embodiments of treating spinal cord injury comprising administration of a population of cells in which one cell type of said populations is a T cell possessing ability to inhibit proliferation of other T cells.

Preferred embodiments include methods wherein said T cell possessing ability to inhibit proliferation of other T cells suppresses cytokine production of other T cells.

Preferred embodiments include methods wherein said suppression of cytokine production of other T cells is reduction of interferon gamma.

Preferred embodiments include methods wherein said suppression of cytokine production of other T cells is reduction of cytokines induced by STAT3 activation.

Preferred embodiments include methods wherein said suppression of cytokine production of other T cells is reduction of cytokines induced by STAT6 activation.

Preferred embodiments include methods wherein said suppression of cytokine production of other T cells is reduction of cytokines induced by NF-kappa B translocation.

Preferred embodiments include methods wherein said suppression of cytokine production of other T cells is reduction of cytokines induced by janus activated kinase phosphorylation.

Preferred embodiments include methods wherein cytokine is interleukin-2.

Preferred embodiments include methods wherein cytokine is interleukin-4.

Preferred embodiments include methods wherein cytokine is interleukin-6.

Preferred embodiments include methods wherein cytokine is interleukin-8.

Preferred embodiments include methods wherein cytokine is interleukin-1.

Preferred embodiments include methods wherein cytokine is interleukin-12.

Preferred embodiments include methods wherein cytokine is interleukin-15.

Preferred embodiments include methods wherein cytokine is interleukin-18.

Preferred embodiments include methods wherein cytokine is interleukin-17.

Preferred embodiments include methods wherein cytokine is interleukin-17C.

Preferred embodiments include methods wherein cytokine is interleukin-21.

Preferred embodiments include methods wherein cytokine is interleukin-23.

Preferred embodiments include methods wherein cytokine is interleukin-27.

Preferred embodiments include methods wherein cytokine is lymphotoxin.

Preferred embodiments include methods wherein cytokine is TNF-alpha.

Preferred embodiments include methods wherein cytokine is TRANCE.

Preferred embodiments include methods wherein cytokine is BLyS.

Preferred embodiments include methods wherein said T cell capable of suppressing proliferation of other T cells is capable of enhancing production of TGF-beta from said other T cells.

Preferred embodiments include methods wherein said T cell capable of suppressing proliferation of other T cells is capable of enhancing production of IL-10 from said other T cells.

Preferred embodiments include methods wherein said T cell capable of suppressing proliferation of other T cells is capable of enhancing production of VEGF from said other T cells.

Preferred embodiments include methods wherein said T cell capable of suppressing proliferation of other T cells is capable of enhancing production of HLA-G from said other T cells.

Preferred embodiments include methods wherein said T cell capable of suppressing proliferation of other T cells is capable of enhancing production of interleukin-1 receptor antagonist from said other T cells.

Preferred embodiments include methods wherein said T cell capable of suppressing proliferation of other T cells is capable of enhancing production of angiopoietin from said other T cells.

Preferred embodiments include methods wherein said T cell capable of suppressing proliferation of other T cells is capable of enhancing production of VEGF from said other T cells.

Preferred embodiments include methods wherein said T cell capable of suppressing proliferation of other T cells is capable of enhancing production of HGF from said other T cells.

Preferred embodiments include methods wherein said T cell capable of suppressing proliferation of other T cells is a T regulatory cell.

Preferred embodiments include methods wherein said T regulatory cell expresses CD25.

Preferred embodiments include methods wherein said T regulatory cell expresses CTLA4.

Preferred embodiments include methods wherein said T regulatory cell expresses membrane bound TGF-beta.

Preferred embodiments include methods wherein said T regulatory cell expresses Fas Ilgand.

Preferred embodiments include methods wherein said T regulatory cell expresses perforin.

Preferred embodiments include methods wherein said T regulatory cell expresses granzyme B.

Preferred embodiments include methods wherein said T regulatory cell expresses CD73.

Preferred embodiments include methods wherein said T regulatory cell expresses CD105.

Preferred embodiments include methods wherein said T regulatory cell expresses FoxP3.

Preferred embodiments include methods wherein said T regulatory cell expresses CD39.

Preferred embodiments include methods wherein said T regulatory cell expresses endoglin.

Preferred embodiments include methods wherein said T regulatory cell expresses GITR.

Preferred embodiments include methods wherein said T regulatory cell expresses LAG3.

Preferred embodiments include methods wherein said T regulatory cell expresses CD69.

Preferred embodiments include methods wherein said T regulatory cell expresses LRRC32.

Preferred embodiments include methods wherein said T regulatory cell expresses neuropilin.

Preferred embodiments include methods wherein said T regulatory cell is generated from placental tissues.

Preferred embodiments include methods wherein said T regulatory cells are isolated by cutting placental tissue into pieces approximately 1-5 millimeters, digesting said tissue with a mixture of dissociative enzymes, filtering said cells to remove tissue aggregates and culturing in a liquid media.

Preferred embodiments include methods wherein said dissociative enzyme is trypsin.

Preferred embodiments include methods wherein said dissociative enzyme is collagenase.

Preferred embodiments include methods wherein said dissociative enzyme is gelatinase.

Preferred embodiments include methods wherein said liquid media is AIM-V media.

Preferred embodiments include methods wherein said liquid media is supplemented with mitogens selective for T regulatory cells.

Preferred embodiments include methods wherein said mitogen selective for T regulatory cells is interleukin-2 together with TGF-beta.

Preferred embodiments include methods wherein said mitogen selective for T regulatory cells is interleukin-2 together with interleukin-10.

Preferred embodiments include methods wherein said mitogen selective for T regulatory cells is interleukin-2 together with interleukin-10 and TGF-beta.

Preferred embodiments include methods wherein said mitogen selective for T regulatory cells is interleukin-2 together with anti-CD3 antibody.

Preferred embodiments include methods wherein said mitogen selective for T regulatory cells is interleukin-2 together with anti-CD3 antibody together with TGF-beta.

Preferred embodiments include methods wherein said mitogen selective for T regulatory cells is interleukin-2 together with anti-CD3 antibody and with interleukin-10.

Preferred embodiments include methods wherein said mitogen selective for T regulatory cells is interleukin-2 together with anti-CD3 antibody, TGF-beta, and interleukin-10.

Preferred embodiments include methods wherein said T regulatory cell is isolated from umbilical cord blood.

Preferred embodiments include methods wherein said T regulatory cell is generated by culture of umbilical cord blood CD34 expressing progenitor cells with allogeneic mesenchymal stem cells.

Preferred embodiments include methods wherein said T regulatory cell is generated by culture of umbilical cord blood CD34 expressing progenitor cells with allogeneic immature dendritic cells.

Preferred embodiments include methods wherein said immature dendritic cells express lower levels of CD80 as compared to mature dendritic cells.

Preferred embodiments include methods wherein said immature dendritic cells express lower levels of CD86 as compared to mature dendritic cells.

Preferred embodiments include methods wherein said immature dendritic cells express lower levels of CD40 as compared to mature dendritic cells.

Preferred embodiments include methods wherein said immature dendritic cells express lower levels of HLA-II as compared to mature dendritic cells.

Preferred embodiments include methods wherein said immature dendritic cells express lower levels of transporter associated protein-1 as compared to mature dendritic cells.

Preferred embodiments include methods wherein said immature dendritic cells express lower levels of IL-1 as compared to mature dendritic cells.

Preferred embodiments include methods wherein said immature dendritic cells express lower levels of ICAM-1 as compared to mature dendritic cells.

Preferred embodiments include methods wherein said immature dendritic cells express lower levels of LFA-3 as compared to mature dendritic cells.

Preferred embodiments include methods wherein said immature dendritic cells express lower levels of IL-6 as compared to mature dendritic cells.

Preferred embodiments include methods wherein said immature dendritic cells express lower levels of IL-8 as compared to mature dendritic cells.

Preferred embodiments include methods wherein said immature dendritic cells express lower levels of IL-12 as compared to mature dendritic cells.

Preferred embodiments include methods wherein said immature dendritic cells express lower levels of IL-15 as compared to mature dendritic cells.

Preferred embodiments include methods wherein said immature dendritic cells express lower levels of IL-17 as compared to mature dendritic cells.

Preferred embodiments include methods wherein said immature dendritic cells express lower levels of IL-18 as compared to mature dendritic cells.

Preferred embodiments include methods wherein said immature dendritic cells express lower levels of IL-21 as compared to mature dendritic cells.

Preferred embodiments include methods wherein said immature dendritic cells express lower levels of IL-22 as compared to mature dendritic cells.

Preferred embodiments include methods wherein said immature dendritic cells express lower levels of IL-23 as compared to mature dendritic cells.

Preferred embodiments include methods wherein said immature dendritic cells express lower levels of IL-27 as compared to mature dendritic cells.

Preferred embodiments include methods wherein said immature dendritic cells express lower levels of IL-33 as compared to mature dendritic cells.

Preferred embodiments include methods wherein said immature dendritic cells express lower levels of HMGB-1 as compared to mature dendritic cells.

Preferred embodiments include methods wherein said immature dendritic cells express lower levels of toll like receptor 3 as compared to mature dendritic cells.

Preferred embodiments include methods wherein said immature dendritic cells express lower levels of toll like receptor 4 as compared to mature dendritic cells.

Preferred embodiments include methods wherein said immature dendritic cells express lower levels of toll like receptor 5 as compared to mature dendritic cells.

Preferred embodiments include methods wherein said immature dendritic cells express lower levels of toll like receptor 7 as compared to mature dendritic cells.

Preferred embodiments include methods wherein said immature dendritic cells express lower levels of toll like receptor 9 as compared to mature dendritic cells.

Preferred embodiments include methods wherein said immature dendritic cells express lower levels of RIG-1 as compared to mature dendritic cells.

Preferred embodiments include methods wherein said immature dendritic cells express lower levels of MDA5 3 as compared to mature dendritic cells.

Preferred embodiments include methods wherein said immature dendritic cells express lower levels of AIM2 as compared to mature dendritic cells.

Preferred embodiments include methods wherein said immature dendritic cells express higher levels of IL-10 as compared to mature dendritic cells.

Preferred embodiments include methods wherein said immature dendritic cells express higher levels of IL-3 as compared to mature dendritic cells.

Preferred embodiments include methods wherein said immature dendritic cells express higher levels of IL-4 as compared to mature dendritic cells.

Preferred embodiments include methods wherein said immature dendritic cells express higher levels of IL-13 as compared to mature dendritic cells.

Preferred embodiments include methods wherein said immature dendritic cells express higher levels of TGF-beta as compared to mature dendritic cells.

Preferred embodiments include methods wherein said immature dendritic cells express higher levels of TIM3 as compared to mature dendritic cells.

Preferred embodiments include methods wherein said immature dendritic cells express higher levels of IL-12 p40 homodimer as compared to mature dendritic cells.

Preferred embodiments include methods wherein said immature dendritic cells express higher levels of LAG3 as compared to mature dendritic cells.

Preferred embodiments include methods wherein said immature dendritic cells express higher levels of LIF as compared to mature dendritic cells.

Preferred embodiments include methods wherein said immature dendritic cells express higher levels of HLA-G as compared to mature dendritic cells.

Preferred embodiments include methods wherein said immature dendritic cells express higher levels of ILT3 as compared to mature dendritic cells.

Preferred embodiments include methods wherein said immature dendritic cells express higher levels of arginase as compared to mature dendritic cells.

Preferred embodiments include methods wherein said immature dendritic cells express higher levels of indolamine 2,3 dioxygenase as compared to mature dendritic cells.

Preferred embodiments include methods wherein said immature dendritic cells express higher levels of inhibitor of kappa B as compared to mature dendritic cells.

Preferred embodiments include methods wherein said immature dendritic cells express higher levels of galectin-3 as compared to mature dendritic cells.

Preferred embodiments include methods wherein said immature dendritic cells express higher levels of galectin-9 as compared to mature dendritic cells.

Preferred embodiments include methods wherein said immature dendritic cells express higher levels of CXCR4 as compared to mature dendritic cells.

Preferred embodiments include methods wherein said immature dendritic cells express higher levels of MIP-1 alpha as compared to mature dendritic cells.

Preferred embodiments include methods wherein immature dendritic cells are generated by culture of monocytes in GM-CSF and IL-4 for a period of 2-19 days.

Preferred embodiments include methods wherein immature dendritic cells are generated by culture of monocytes in GM-CSF and IL-4 for a period of 4-15 days.

Preferred embodiments include methods wherein immature dendritic cells are generated by culture of monocytes in GM-CSF and IL-4 for a period of 5-10 days.

Preferred embodiments include methods wherein immature dendritic cells are generated by culture of monocytes in GM-CSF and IL-4 for a period of 7-8 days.

Preferred embodiments include methods wherein immature dendritic cells are generated by culture of monocytes in GM-CSF for a period of 2-19 days.

Preferred embodiments include methods wherein immature dendritic cells are generated by culture of monocytes in GM-CSF for a period of 4-15 days.

Preferred embodiments include methods wherein immature dendritic cells are generated by culture of monocytes in GM-CSF for a period of 5-10 days.

Preferred embodiments include methods wherein immature dendritic cells are generated by culture of monocytes in GM-CSF for a period of 7-8 days.

Preferred embodiments include methods wherein immature dendritic cells are generated by culture of monocytes in GM-CSF, IL-4 and IL-10 for a period of 2-19 days.

Preferred embodiments include methods wherein immature dendritic cells are generated by culture of monocytes in GM-CSF, IL-4 and IL-10 for a period of 4-15 days.

Preferred embodiments include methods wherein immature dendritic cells are generated by culture of monocytes in GM-CSF, IL-4 and IL-10 for a period of 5-10 days.

Preferred embodiments include methods wherein immature dendritic cells are generated by culture of monocytes in GM-CSF, IL-4 and IL-10 for a period of 7-8 days.

Preferred embodiments include methods wherein immature dendritic cells are generated by culture of monocytes in GM-CSF, IL-4 and TGF-beta for a period of 2-19 days.

Preferred embodiments include methods wherein immature dendritic cells are generated by culture of monocytes in GM-CSF, IL-4 and TGF-beta for a period of 4-15 days.

Preferred embodiments include methods wherein immature dendritic cells are generated by culture of monocytes in GM-CSF, IL-4 and TGF-beta for a period of 5-10 days.

Preferred embodiments include methods wherein immature dendritic cells are generated by culture of monocytes in GM-CSF, IL-4 and TGF-beta for a period of 7-8 days.

Preferred embodiments include methods wherein immature dendritic cells are generated by culture of monocytes in GM-CSF, IL-4 and VEGF for a period of 2-19 days.

Preferred embodiments include methods wherein immature dendritic cells are generated by culture of monocytes in GM-CSF, IL-4 and VEGF for a period of 4-15 days.

Preferred embodiments include methods wherein immature dendritic cells are generated by culture of monocytes in GM-CSF, IL-4 and VEGF for a period of 5-10 days.

Preferred embodiments include methods wherein immature dendritic cells are generated by culture of monocytes in GM-CSF, IL-4 and VEGF for a period of 7-8 days.

Preferred embodiments include methods wherein immature dendritic cells are generated by culture of monocytes in GM-CSF, IL-4 and PGE2 for a period of 2-19 days.

Preferred embodiments include methods wherein immature dendritic cells are generated by culture of monocytes in GM-CSF, IL-4 and PGE2 for a period of 4-15 days.

Preferred embodiments include methods wherein immature dendritic cells are generated by culture of monocytes in GM-CSF, IL-4 and PGE2 for a period of 5-10 days.

Preferred embodiments include methods wherein immature dendritic cells are generated by culture of monocytes in GM-CSF, IL-4 and PGE2 for a period of 7-8 days.

Preferred embodiments include methods wherein immature dendritic cells are generated by culture of monocytes in GM-CSF, IL-4 and IL-35 for a period of 2-19 days.

Preferred embodiments include methods wherein immature dendritic cells are generated by culture of monocytes in GM-CSF, IL-4 and IL-35 for a period of 4-15 days.

Preferred embodiments include methods wherein immature dendritic cells are generated by culture of monocytes in GM-CSF, IL-4 and IL-35 for a period of 5-10 days.

Preferred embodiments include methods wherein immature dendritic cells are generated by culture of monocytes in GM-CSF, IL-4 and IL-35 for a period of 7-8 days.

Preferred embodiments include methods wherein said T regulatory cell is isolated from peripheral blood.

Preferred embodiments include methods wherein said T regulatory cell is generated from peripheral blood that was extracted subsequent to administration of the donor an agent capable of mobilizing T regulatory cells and/or T regulatory cell progenitors.

Preferred embodiments include methods wherein said T regulatory cell progenitors express CD34.

Preferred embodiments include methods wherein said T regulatory cell progenitors express CD25.

Preferred embodiments include methods wherein said T regulatory cell progenitors express CD133.

Preferred embodiments include methods wherein said T regulatory cell progenitors express c-kit.

Preferred embodiments include methods wherein said T regulatory cell progenitors express FoxP3.

Preferred embodiments include methods wherein said T regulatory cell progenitors express NANOG.

Preferred embodiments include methods wherein said T regulatory cell progenitors express CD34 and NANOG.

Preferred embodiments include methods wherein said T regulatory cell progenitors express CD34 and CD25.

Preferred embodiments include methods wherein said T regulatory cell progenitors express CD73.

Preferred embodiments include methods wherein said T regulatory cell progenitors express CD34 and CD73.

Preferred embodiments include methods wherein said T regulatory cell progenitors express CD34 and FoxP3.

Preferred embodiments include methods wherein said T regulatory cell progenitors express neuropilin.

Preferred embodiments include methods wherein said T regulatory cell progenitors express CD34 and neuropilin.

Preferred embodiments include methods wherein said T regulatory cell progenitors express CD133 and FoxP3.

Preferred embodiments include methods wherein said T regulatory cell progenitors express CD133 and neuropilin.

Preferred embodiments include methods wherein said T regulatory cell progenitors express CD133 and c-kit.

Preferred embodiments include methods wherein said T regulatory cell progenitors express CD133 and CD25.

Preferred embodiments include methods wherein said T regulatory cell progenitors express CD133 and CTLA4.

Preferred embodiments include methods wherein said T regulatory cell progenitors express CD133 and LAG-3.

Preferred embodiments include methods wherein said T regulatory cell progenitors express CD133 and TIM-3.

Preferred embodiments include methods wherein said T regulatory cell progenitors express CD133 and membrane TGF-beta.

Preferred embodiments include methods wherein said T regulatory cell progenitors are mobilized by treatment of the donor with interleukin-2.

Preferred embodiments include methods wherein said T regulatory cell progenitors are mobilized by treatment of the donor with cyclophosphamide.

Preferred embodiments include methods wherein said T regulatory cell progenitors are mobilized by treatment of the donor with 5 fluorouracil.

Preferred embodiments include methods wherein said T regulatory cell progenitors are mobilized by treatment of the donor with doxorubicin.

Preferred embodiments include methods wherein said T regulatory cell progenitors are mobilized by treatment of the donor with G-CSF.

Preferred embodiments include methods wherein said T regulatory cell progenitors are mobilized by treatment of the donor with interleukin-1 beta.

Preferred embodiments include methods wherein said T regulatory cell progenitors are mobilized by treatment of the donor with interleukin-7.

Preferred embodiments include methods wherein said T regulatory cell progenitors are mobilized by treatment of the donor with interleukin-10.

Preferred embodiments include methods wherein said T regulatory cell progenitors are mobilized by treatment of the donor with interferon gamma.

Preferred embodiments include methods wherein said T regulatory cell progenitors are mobilized by treatment of the donor with interferon alpha.

Preferred embodiments include methods wherein said T regulatory cell progenitors are mobilized by treatment of the donor with interferon beta.

Preferred embodiments include methods wherein said T regulatory cell progenitors are mobilized by treatment of the donor with interferon omega.

Preferred embodiments include methods wherein said T regulatory cell progenitors are mobilized by treatment of the donor with interferon tau.

Preferred embodiments include methods wherein said T regulatory cell progenitors are mobilized by treatment of the donor with G-CSF.

Preferred embodiments include methods wherein said T regulatory cell progenitors are mobilized by treatment of the donor with M-CSF.

Preferred embodiments include methods wherein said T regulatory cell progenitors are mobilized by treatment of the donor with interferon GM-CSF.

Preferred embodiments include methods wherein said T regulatory cell progenitors are mobilized by treatment of the donor with cyclodextrin.

Preferred embodiments include methods wherein said T regulatory cell progenitors are mobilized by treatment of the donor with BCG.

Preferred embodiments include methods wherein said T regulatory cell progenitors are mobilized by treatment of the donor with cyclodextrin.

Preferred embodiments include methods wherein said T regulatory cell progenitors are mobilized by treatment of the donor with flagellin.

Preferred embodiments include methods wherein said T regulatory cell progenitors are mobilized by treatment of the donor with onconase.

Preferred embodiments include methods wherein said T regulatory cell progenitors are mobilized by treatment of the donor with Poly IC.

Preferred embodiments include methods wherein said T regulatory cell progenitors are mobilized by treatment of the donor with Poly LC.

Preferred embodiments include methods wherein said T regulatory cell progenitors are mobilized by treatment of the donor with human chorionic gonadotropin.

Preferred embodiments include methods wherein said T regulatory cell progenitors are mobilized by treatment of the donor with oxytocin.

Preferred embodiments include methods wherein said T regulatory cell progenitors are mobilized by treatment of the donor with thymosin beta 4.

Preferred embodiments include methods wherein said T regulatory cell progenitors are mobilized by treatment of the donor with hepatocyte growth factor.

Preferred embodiments include methods wherein said T regulatory cell progenitors are mobilized by treatment of the donor with a proteosome inhibitor.

Preferred embodiments include methods wherein said proteosome inhibitor is velcade.

Preferred embodiments include methods wherein said T regulatory cell progenitors are mobilized by treatment of the donor with GITR ligand.

Preferred embodiments include methods wherein said T regulatory cell progenitors are mobilized by treatment of the donor with anti-CD3 antibody.

Preferred embodiments include methods wherein said T regulatory cell progenitors are mobilized by treatment of the donor with anti-CTLA4.

Preferred embodiments include methods wherein said T regulatory cell progenitors are mobilized by treatment of the donor with anti-CD45RB antibody.

Preferred embodiments include methods wherein said T regulatory cell progenitors are mobilized by treatment of the donor with VEGF.

Preferred embodiments include methods wherein said T regulatory cell progenitors are mobilized by treatment of the donor with IGF.

Preferred embodiments include methods wherein said T regulatory cell progenitors are mobilized by treatment of the donor with placental growth factor.

Preferred embodiments include methods wherein said T regulatory cell progenitors are mobilized by treatment of the donor with intravenous ascorbic acid.

Preferred embodiments include methods wherein said T regulatory cell progenitors are mobilized by treatment of the donor with intravenous apatone.

Preferred embodiments include methods wherein said T regulatory cell progenitors are mobilized by treatment of the donor with a PD-L1 inhibitor.

Preferred embodiments include methods wherein said PD-L1 inhibitor is an antibody.

Preferred embodiments include methods wherein said PD-L1 inhibitor is a siRNA molecule.

Preferred embodiments include methods wherein said PD-L1 inhibitor is a shRNA molecule.

Preferred embodiments include methods wherein said PD-L1 inhibitor is a ribozyme.

Preferred embodiments include methods wherein said PD-L1 inhibitor is a small molecule.

Preferred embodiments include methods wherein said PD-L1 inhibitor is an aptamer.

Preferred embodiments include methods wherein said PD-L1 inhibitor is a hammerhead ribozyme.

Preferred embodiments include methods wherein said T regulatory cell is isolated from menstrual blood.

Preferred embodiments include methods wherein said T regulatory cell is isolated from cerebral spinal fluid.

Preferred embodiments include methods wherein said T regulatory cell is isolated from omental tissue.

Preferred embodiments include methods wherein said T regulatory cell is isolated from adipose tissue.

Preferred embodiments include methods wherein said T regulatory cell is isolated from bone marrow.

Preferred embodiments include methods wherein said T regulatory cell is isolated from bone marrow of a patient treated with beta glucan.

Preferred embodiments include methods wherein said T regulatory cell is isolated from bone marrow of a patient treated with G-CSF.

Preferred embodiments include methods wherein said T regulatory cell is isolated from bone marrow of a patient treated with M-CSF.

Preferred embodiments include methods wherein said T regulatory cell is isolated from bone marrow of a patient treated with GM-CSF.

Preferred embodiments include methods wherein said T regulatory cell is isolated from bone marrow of a patient treated with flt-3 ligand.

Preferred embodiments include methods wherein said T regulatory cells are generated from pluripotent stem cells.

Preferred embodiments include methods wherein said pluripotent stem cells are generated by mechanical stress.

Preferred embodiments include methods wherein said pluripotent stem cells are generated by somatic cell nuclear transfer.

Preferred embodiments include methods wherein said pluripotent stem cells are generated by parthenogenesis.

Preferred embodiments include methods wherein said pluripotent stem cells are generated from embryonic stem cells.

Preferred embodiments include methods wherein said pluripotent stem cells are generated from induced pluripotent stem cells.

Preferred embodiments include methods wherein said induced pluripotent stem cells are generated as a clonal population.

Preferred embodiments include methods wherein said induced pluripotent stem cells are generated by administration of a retroviral expression vector into cell form which said induced pluripotent stem cells are to be derived, wherein said retroviral expression vector contains one or more dedifferentiating factors.

Preferred embodiments include methods wherein said dedifferentiating factor is SOX-2.

Preferred embodiments include methods wherein said dedifferentiating factor is c-met.

Preferred embodiments include methods wherein said dedifferentiating factor is OCT4.

Preferred embodiments include methods wherein said dedifferentiating factor is Jhdmd1b.

Preferred embodiments include methods wherein said dedifferentiating factor is KLF4.

Preferred embodiments include methods wherein said dedifferentiating factor is Jhdmd1b.

Preferred embodiments include methods wherein said cell to be used for generation of said induced pluripotent stem cells is synchronized in cell cycle.

Preferred embodiments include methods wherein said synchronization is achieved by exposure to a microtubule inhibitor.

Preferred embodiments include methods wherein said cell to be used for generation of said induced pluripotent stem cells is a hepatocyte.

Preferred embodiments include methods wherein said cell to be used for generation of said induced pluripotent stem cells is a fibroblast.

Preferred embodiments include methods wherein said fibroblast is derived from the bone marrow.

Preferred embodiments include methods wherein said fibroblast is derived from adipose tissue.

Preferred embodiments include methods wherein said fibroblast is derived from peripheral blood.

Preferred embodiments include methods wherein said fibroblast is derived from mobilized peripheral blood.

Preferred embodiments include methods wherein said fibroblast is derived skin cells.

Preferred embodiments include methods wherein said fibroblast is derived from foreskin

Preferred embodiments include methods wherein said fibroblast is derived from endometrium.

Preferred embodiments include methods wherein said fibroblast is derived from fallopian tube.

Preferred embodiments include methods wherein said fibroblast is derived from hair follicle.

Preferred embodiments include methods wherein said fibroblast is derived from testicular tissue.

Preferred embodiments include methods wherein said fibroblast is derived from the omental tissue.

Preferred embodiments include methods wherein said fibroblast is derived from Wharton's Jelly.

Preferred embodiments include methods wherein said cell to be used for generation of said induced pluripotent stem cells is a thymic medullary epithelial cell.

Preferred embodiments include methods wherein said cell to be used for generation of said induced pluripotent stem cells is a thymus derived cell.

Preferred embodiments include methods wherein said cell to be used for generation of said induced pluripotent stem cells is a mesenchymal stem cell.

Preferred embodiments include methods wherein said mesenchymal stem cell has been pretreated with KLOTHO.

Preferred embodiments include methods wherein said mesenchymal stem cell has been pretreated with GDF11.

Preferred embodiments include methods wherein said mesenchymal stem cell has been pretreated with GDF15.

Preferred embodiments include methods wherein said mesenchymal stem cell has been pretreated with BMP2.

Preferred embodiments include methods wherein said mesenchymal stem cell has been pretreated with BMP4.

Preferred embodiments include methods wherein said mesenchymal stem cell has been pretreated with one or more histone deacetylase inhibitors.

Preferred embodiments include methods wherein said histone deacetylase inhibitor is valproic acid.

Preferred embodiments include methods wherein said histone deacetylase inhibitor is trichostatin A.

Preferred embodiments include methods wherein said histone deacetylase inhibitor is entinostat.

Preferred embodiments include methods wherein said histone deacetylase inhibitor is sulforaphane.

Preferred embodiments include methods wherein said histone deacetylase inhibitor is phenylbutyrate.

Preferred embodiments include methods wherein said histone deacetylase inhibitor is Vorinostat.

Preferred embodiments include methods wherein said histone deacetylase inhibitor is Romidepsin.

Preferred embodiments include methods wherein said histone deacetylase inhibitor is Panobinostat.

Preferred embodiments include methods wherein said mesenchymal stem cell has been pretreated with one or more histone GSK-3 inhibitors.

Preferred embodiments include methods wherein said GSK-3 inhibitor is lithium or a salt thereof.

Preferred embodiments include methods wherein said mesenchymal stem cell has been pretreated with one or more histone DNA methyltransferase inhibitors.

Preferred embodiments include methods wherein said DNA methyltransferase inhibitor is gemcitabine.

Preferred embodiments include methods wherein said mesenchymal stem cell has been pretreated with an NF-kappa B inhibitor.

Preferred embodiments include methods wherein said mesenchymal stem cell has been pretreated with a stimulator of HIF-1 alpha.

Preferred embodiments include methods wherein said mesenchymal stem cell has been pretreated with valproic acid, lithium, and sodium phenylbutyrate.

Preferred embodiments include methods wherein said mesenchymal stem cell has been pretreated with a ROCK inhibitor.

Preferred embodiments include methods wherein said mesenchymal stem cell has been pretreated with cytoplasm from a dedifferentiated, or undifferentiated cell.

Preferred embodiments include methods wherein said dedifferentiated or undifferentiated cell is a hematopoietic stem cell.

Preferred embodiments include methods wherein said hematopoietic stem cell expresses NANOG.

Preferred embodiments include methods wherein said hematopoietic stem cell expresses CD34.

Preferred embodiments include methods wherein said hematopoietic stem cell expresses CD133.

Preferred embodiments include methods wherein said hematopoietic stem cell does not express CD38.

Preferred embodiments include methods wherein said hematopoietic stem cell does not express CD14.

Preferred embodiments include methods wherein said hematopoietic stem cell does not express CD16.

Preferred embodiments include methods wherein said hematopoietic stem cell does not express CD24.

Preferred embodiments include methods wherein said hematopoietic stem cell expresses CD105.

Preferred embodiments include methods wherein said hematopoietic stem cell expresses c-met.

Preferred embodiments include methods wherein said hematopoietic stem cell expresses LIF receptor.

Preferred embodiments include methods wherein said hematopoietic stem cell expresses IL-3 receptor.

Preferred embodiments include methods wherein said hematopoietic stem cell expresses NOTCH.

Preferred embodiments include methods wherein said dedifferentiated or undifferentiated cell is a mesenchymal stem cell.

Preferred embodiments include methods wherein said dedifferentiated or undifferentiated cell is a monocyte treated with a histone deacetylase inhibitor.

Preferred embodiments include methods wherein said dedifferentiated or undifferentiated cell is a Sertoli cell.

Preferred embodiments include methods wherein said dedifferentiated or undifferentiated cell is an oocyte.

Preferred embodiments include methods wherein said dedifferentiated or undifferentiated cell is an induced pluripotent stem cell.

Preferred embodiments include methods wherein said retroviral expression vector is a pMXs vector.

Preferred embodiments include methods wherein said retroviral expression vector is a lentiviral vector.

Preferred embodiments include methods wherein said retroviral expression vector is an adenoviral vector.

Preferred embodiments include methods wherein said retroviral expression vector is a pMXs adeno-associated vector.

Preferred embodiments include methods wherein said retroviral expression vector is a herpesvirus vector.

Preferred embodiments include methods wherein said dedifferentiation genes are delivered by means of a plasmid based vector.

Preferred embodiments include methods wherein said dedifferentiation genes are delivered by means of an RNA based vector.

Preferred embodiments include methods wherein said dedifferentiation genes are delivered by means of an mRNA based vector.

Preferred embodiments include methods wherein said dedifferentiation genes are delivered by means of an microRNA based vector.

Preferred embodiments include methods wherein said dedifferentiation factors are administered together with ascorbic acid.

Preferred embodiments include methods wherein said dedifferentiation factors are administered together with ascorbic acid.

Preferred embodiments include methods wherein said dedifferentiation factors are administered together with a stimulator of the RAS pathway.

Preferred embodiments include methods wherein said dedifferentiation factors are administered together with a stimulator of the myc pathway.

Preferred embodiments include methods wherein said dedifferentiation factors are administered together with a stimulator of the PIM-1 pathway.

Preferred embodiments include methods wherein said dedifferentiation factors are administered together with a stimulator of the janus activated kinase pathway.

Preferred embodiments include methods of reducing fibrosis in a patient with spinal cord injury comprising administration of T regulatory cells in the area of injury.

Preferred embodiments include methods wherein said T regulatory cells are administered subsequent to administration of mesenchymal stem cells.

Preferred embodiments include methods wherein said mesenchymal stem cells are selected for release of brain derived neurotrophic factor upon stimulation with molecular signals associated with tissue injury.

Preferred embodiments include methods wherein mesenchymal stem cells are selected for expression of CD56.

Preferred embodiments include methods wherein mesenchymal stem cells produce at least 10 pg of brain derived neurotrophic factor per milliliter subsequent to stimulation with Poly IC.

Preferred embodiments include methods wherein mesenchymal stem cells produce at least 25 pg of brain derived neurotrophic factor per milliliter subsequent to stimulation with Poly IC.

Preferred embodiments include methods wherein mesenchymal stem cells produce at least 100 pg of brain derived neurotrophic factor per milliliter subsequent to stimulation with Poly IC.

Preferred embodiments include methods wherein said mesenchymal stem cells are selected for release of ciliary neurotrophic growth factor upon stimulation with molecular signals associated with tissue injury.

Preferred embodiments include methods wherein mesenchymal stem cells are selected for expression of CD73.

Preferred embodiments include methods wherein mesenchymal stem cells produce at least 4 pg of ciliary neurotrophic growth factor per milliliter subsequent to stimulation with Poly IC.

Preferred embodiments include methods wherein mesenchymal stem cells produce at least 8 pg of ciliary neurotrophic growth factor per milliliter subsequent to stimulation with Poly IC.

Preferred embodiments include methods wherein mesenchymal stem cells produce at least 12 pg of ciliary neurotrophic growth factor per milliliter subsequent to stimulation with Poly IC.

Preferred embodiments include methods wherein said mesenchymal stem cells are selected for release of basic fibroblast growth factor upon stimulation with molecular signals associated with tissue injury.

Preferred embodiments include methods wherein mesenchymal stem cells are selected for expression of CD105.

Preferred embodiments include methods wherein mesenchymal stem cells produce at least 50 pg of basic fibroblast growth factor per milliliter subsequent to stimulation with Poly IC.

Preferred embodiments include methods wherein mesenchymal stem cells produce at least 100 pg of basic fibroblast growth factor per milliliter subsequent to stimulation with Poly IC.

Preferred embodiments include methods wherein mesenchymal stem cells produce at least 100 pg of basic fibroblast growth factor per milliliter subsequent to stimulation with Poly IC.

Preferred embodiments include methods wherein said T regulatory cells are administered subsequent to administration of a fibrinolytic enzyme.

Preferred embodiments include methods wherein said enzyme is a matrix metalloprotease.

Preferred embodiments include methods wherein said matrix metalloprotease is MMP-3.

Preferred embodiments include methods wherein said matrix metalloprotease is MMP-5.

Preferred embodiments include methods wherein said matrix metalloprotease is MMP-6.

Preferred embodiments include methods wherein said matrix metalloprotease is MMP-9.

Preferred embodiments include methods wherein said matrix metalloprotease is MMP-12.

Preferred embodiments include methods wherein said T regulatory cells are administered subsequent to administration of an inhibitor of NOGO.

Preferred embodiments include methods wherein said inhibitor of NOGO is an antisense oligonucleotide.

Preferred embodiments include methods wherein said inhibitor of NOGO is a one or more molecules capable of inducing RNA interference.

Preferred embodiments include methods wherein said molecule capable of inducing RNA interference is a short interfering RNA.

Preferred embodiments include methods wherein said molecule capable of inducing RNA interference is a short hairpin RNA.

Preferred embodiments include methods wherein said inhibitor of NOGO is a small molecule inhibitor.

Preferred embodiments include methods wherein said inhibitor of NOGO is an antibody.

Preferred embodiments include methods wherein said inhibitor of NOGO is an aptamer.

Preferred embodiments include methods wherein said inhibitor of NOGO is a somamer.

Preferred embodiments include methods wherein said inhibitor of NOGO is bispecific antibody.

Preferred embodiments include methods wherein said inhibitor of NOGO is a ribozyme.

Preferred embodiments include methods wherein said inhibitor of NOGO is an antibody.

Preferred embodiments include methods wherein said inhibitor of NOGO is a microantibody.

Preferred embodiments include methods wherein said inhibitor of NOGO is a hammerhead ribozyme.

Preferred embodiments include methods wherein said inhibitor of NOGO is a soluble receptor.

Preferred embodiments include methods wherein said T regulatory cells are administered subsequent to administration of a myeloid lineage cell.

Preferred embodiments include methods wherein said myeloid lineage cell is a myeloid derived dendritic cell.

Preferred embodiments include methods wherein said myeloid dendritic cell is immature.

Preferred embodiments include methods wherein said myeloid dendritic cell generated from a monocytic progenitor.

Preferred embodiments include methods wherein said monocyte is isolated from peripheral blood.

Preferred embodiments include methods wherein said monocyte is isolated from peripheral blood that is mobilized by treatment with G-CSF.

Preferred embodiments include methods wherein said monocyte is isolated from peripheral blood that is mobilized by treatment with GM-CSF.

Preferred embodiments include methods wherein said monocyte is isolated from peripheral blood that is mobilized by treatment with M-CSF.

Preferred embodiments include methods wherein said monocyte is isolated from peripheral blood that is mobilized by treatment with interleukin-3.

Preferred embodiments include methods wherein said monocyte is isolated from peripheral blood that is mobilized by treatment with interleukin-10.

Preferred embodiments include methods wherein said monocyte is isolated from peripheral blood that is mobilized by treatment with beta glucan.

Preferred embodiments include methods wherein said monocyte is isolated from peripheral blood that is mobilized by treatment with interferon gamma.

Preferred embodiments include methods wherein said monocyte is isolated from menstrual blood.

Preferred embodiments include methods wherein said monocyte is isolated from umbilical cord blood.

Preferred embodiments include methods wherein said monocyte is isolated from Wharton's Jelly.

Preferred embodiments include methods wherein said monocyte is isolated from bone marrow.

Preferred embodiments include methods wherein said monocyte is isolated from adipose tissue.

Preferred embodiments include methods wherein said monocyte is isolated from omental tissue.

Preferred embodiments include methods wherein said monocyte is pretreated with one or more agents to induce a tolerogenic phenotype prior to differentiation into a myeloid dendritic cells with immature phenotype.

Preferred embodiments include methods wherein said monocyte is pretreated with PGE2 at a sufficient concentration and for a time period to stimulate production of at least 10 pg/ml of interleukin-10 from a culture of 1 million monocytes.

Preferred embodiments include methods wherein said monocyte is pretreated with PGE2 at a sufficient concentration and for a time period to stimulate production of at least 20 pg/ml of interleukin-10 from a culture of 1 million monocytes.

Preferred embodiments include methods wherein said monocyte is pretreated with PGE2 at a sufficient concentration and for a time period to stimulate production of at least 40 pg/ml of interleukin-10 from a culture of 1 million monocytes.

Preferred embodiments include methods wherein said monocyte is pretreated with genistein at a sufficient concentration and for a time period to stimulate production of at least 10 pg/ml of interleukin-10 from a culture of 1 million monocytes.

Preferred embodiments include methods wherein said monocyte is pretreated with genistein at a sufficient concentration and for a time period to stimulate production of at least 20 pg/ml of interleukin-10 from a culture of 1 million monocytes.

Preferred embodiments include methods wherein said monocyte is pretreated with genistein at a sufficient concentration and for a time period to stimulate production of at least 40 pg/ml of interleukin-10 from a culture of 1 million monocytes.

Preferred embodiments include methods wherein said monocyte is pretreated with quercetin at a sufficient concentration and for a time period to stimulate production of at least 10 pg/ml of interleukin-10 from a culture of 1 million monocytes.

Preferred embodiments include methods wherein said monocyte is pretreated with quercetin at a sufficient concentration and for a time period to stimulate production of at least 20 pg/ml of interleukin-10 from a culture of 1 million monocytes.

Preferred embodiments include methods wherein said monocyte is pretreated with quercetin at a sufficient concentration and for a time period to stimulate production of at least 40 pg/ml of interleukin-10 from a culture of 1 million monocytes.

Preferred embodiments include methods wherein said monocyte is pretreated with hypertonic saline at a sufficient concentration and for a time period to stimulate production of at least 10 pg/ml of interleukin-10 from a culture of 1 million monocytes.

Preferred embodiments include methods wherein said monocyte is pretreated with hypertonic saline at a sufficient concentration and for a time period to stimulate production of at least 20 pg/ml of interleukin-10 from a culture of 1 million monocytes.

Preferred embodiments include methods wherein said monocyte is pretreated with hypertonic saline at a sufficient concentration and for a time period to stimulate production of at least 40 pg/ml of interleukin-10 from a culture of 1 million monocytes.

Preferred embodiments include methods wherein said monocyte is pretreated with VEGF at a sufficient concentration and for a time period to stimulate production of at least 10 pg/ml of interleukin-10 from a culture of 1 million monocytes.

Preferred embodiments include methods wherein said monocyte is pretreated with VEGF at a sufficient concentration and for a time period to stimulate production of at least 20 pg/ml of interleukin-10 from a culture of 1 million monocytes.

Preferred embodiments include methods wherein said monocyte is pretreated with VEGF at a sufficient concentration and for a time period to stimulate production of at least 40 pg/ml of interleukin-10 from a culture of 1 million monocytes.

Preferred embodiments include methods wherein said monocyte is pretreated with PDGF-BB at a sufficient concentration and for a time period to stimulate production of at least 10 pg/ml of interleukin-10 from a culture of 1 million monocytes.

Preferred embodiments include methods wherein said monocyte is pretreated with PDGF-BB at a sufficient concentration and for a time period to stimulate production of at least 20 pg/ml of interleukin-10 from a culture of 1 million monocytes.

Preferred embodiments include methods wherein said monocyte is pretreated with PDGF-BB at a sufficient concentration and for a time period to stimulate production of at least 40 pg/ml of interleukin-10 from a culture of 1 million monocytes.

Preferred embodiments include methods wherein said monocyte is pretreated with IGF-1 at a sufficient concentration and for a time period to stimulate production of at least 10 pg/ml of interleukin-10 from a culture of 1 million monocytes.

Preferred embodiments include methods wherein said monocyte is pretreated with IGF-1 at a sufficient concentration and for a time period to stimulate production of at least 20 pg/ml of interleukin-10 from a culture of 1 million monocytes.

Preferred embodiments include methods wherein said monocyte is pretreated with IGF-1 at a sufficient concentration and for a time period to stimulate production of at least 40 pg/ml of interleukin-10 from a culture of 1 million monocytes.

Preferred embodiments include methods wherein said monocyte is pretreated with salinomycin at a sufficient concentration and for a time period to stimulate production of at least 10 pg/ml of interleukin-10 from a culture of 1 million monocytes.

Preferred embodiments include methods wherein said monocyte is pretreated with salinomycin at a sufficient concentration and for a time period to stimulate production of at least 20 pg/ml of interleukin-10 from a culture of 1 million monocytes.

Preferred embodiments include methods wherein said monocyte is pretreated with salinomycin at a sufficient concentration and for a time period to stimulate production of at least 40 pg/ml of interleukin-10 from a culture of 1 million monocytes.

Preferred embodiments include methods wherein said monocyte is pretreated with erythropoietin at a sufficient concentration and for a time period to stimulate production of at least 10 pg/ml of interleukin-10 from a culture of 1 million monocytes.

Preferred embodiments include methods wherein said monocyte is pretreated with erythropoietin at a sufficient concentration and for a time period to stimulate production of at least 20 pg/ml of interleukin-10 from a culture of 1 million monocytes.

Preferred embodiments include methods wherein said monocyte is pretreated with erythropoietin at a sufficient concentration and for a time period to stimulate production of at least 40 pg/ml of interleukin-10 from a culture of 1 million monocytes.

Preferred embodiments include methods wherein said monocyte is pretreated with NF-kappa B decoy oligonucleotides at a sufficient concentration and for a time period to stimulate production of at least 10 pg/ml of interleukin-10 from a culture of 1 million monocytes.

Preferred embodiments include methods wherein said monocyte is pretreated with NF-kappa B decoy oligonucleotides at a sufficient concentration and for a time period to stimulate production of at least 20 pg/ml of interleukin-10 from a culture of 1 million monocytes.

Preferred embodiments include methods wherein said monocyte is pretreated with NF-kappa B decoy oligonucleotides at a sufficient concentration and for a time period to stimulate production of at least 40 pg/ml of interleukin-10 from a culture of 1 million monocytes.

Preferred embodiments include methods wherein said monocyte is pretreated with Ikk- B decoy oligonucleotides at a sufficient concentration and for a time period to stimulate production of at least 10 pg/ml of interleukin-10 from a culture of 1 million monocytes.

Preferred embodiments include methods wherein said monocyte is pretreated with Ikk-B decoy oligonucleotides at a sufficient concentration and for a time period to stimulate production of at least 20 pg/ml of interleukin-10 from a culture of 1 million monocytes.

Preferred embodiments include methods wherein said monocyte is pretreated with Ikk-B decoy oligonucleotides at a sufficient concentration and for a time period to stimulate production of at least 40 pg/ml of interleukin-10 from a culture of 1 million monocytes.

Preferred embodiments include methods wherein said monocyte is pretreated with interleukin-35 at a sufficient concentration and for a time period to stimulate production of at least 10 pg/ml of interleukin-10 from a culture of 1 million monocytes.

Preferred embodiments include methods wherein said monocyte is pretreated with interleukin-35 at a sufficient concentration and for a time period to stimulate production of at least 20 pg/ml of interleukin-10 from a culture of 1 million monocytes.

Preferred embodiments include methods wherein said monocyte is pretreated with interleukin-35 at a sufficient concentration and for a time period to stimulate production of at least 40 pg/ml of interleukin-10 from a culture of 1 million monocytes.

Preferred embodiments include methods wherein said monocyte is pretreated with TGF-beta at a sufficient concentration and for a time period to stimulate production of at least 10 pg/ml of interleukin-10 from a culture of 1 million monocytes.

Preferred embodiments include methods wherein said monocyte is pretreated with TGF-beta at a sufficient concentration and for a time period to stimulate production of at least 20 pg/ml of interleukin-10 from a culture of 1 million monocytes.

Preferred embodiments include methods wherein said monocyte is pretreated with TGF-beta at a sufficient concentration and for a time period to stimulate production of at least 40 pg/ml of interleukin-10 from a culture of 1 million monocytes.

Preferred embodiments include methods wherein said monocyte is pretreated with ascorbic acid at a sufficient concentration and for a time period to stimulate production of at least 10 pg/ml of interleukin-10 from a culture of 1 million monocytes.

Preferred embodiments include methods wherein said monocyte is pretreated with ascorbic acid at a sufficient concentration and for a time period to stimulate production of at least 20 pg/ml of interleukin-10 from a culture of 1 million monocytes.

Preferred embodiments include methods wherein said monocyte is pretreated with ascorbic acid at a sufficient concentration and for a time period to stimulate production of at least 40 pg/ml of interleukin-10 from a culture of 1 million monocytes.

Preferred embodiments include methods wherein said monocyte is pretreated with n-acetylcysteine at a sufficient concentration and for a time period to stimulate production of at least 10 pg/ml of interleukin-10 from a culture of 1 million monocytes.

Preferred embodiments include methods wherein said monocyte is pretreated with n-acetylcysteine at a sufficient concentration and for a time period to stimulate production of at least 20 pg/ml of interleukin-10 from a culture of 1 million monocytes.

Preferred embodiments include methods wherein said monocyte is pretreated with n-acetylcysteine at a sufficient concentration and for a time period to stimulate production of at least 40 pg/ml of interleukin-10 from a culture of 1 million monocytes.

Preferred embodiments include methods wherein said monocyte is pretreated with alpha lipoic acid at a sufficient concentration and for a time period to stimulate production of at least 10 pg/ml of interleukin-10 from a culture of 1 million monocytes.

Preferred embodiments include methods wherein said monocyte is pretreated with alpha lipoic acid at a sufficient concentration and for a time period to stimulate production of at least 20 pg/ml of interleukin-10 from a culture of 1 million monocytes.

Preferred embodiments include methods wherein said monocyte is pretreated with alpha lipoic acid at a sufficient concentration and for a time period to stimulate production of at least 40 pg/ml of interleukin-10 from a culture of 1 million monocytes.

Preferred embodiments include methods wherein said monocyte is pretreated with methylene blue at a sufficient concentration and for a time period to stimulate production of at least 10 pg/ml of interleukin-10 from a culture of 1 million monocytes.

Preferred embodiments include methods wherein said monocyte is pretreated with methylene blue at a sufficient concentration and for a time period to stimulate production of at least 20 pg/ml of interleukin-10 from a culture of 1 million monocytes.

Preferred embodiments include methods wherein said monocyte is pretreated with methylene blue at a sufficient concentration and for a time period to stimulate production of at least 40 pg/ml of interleukin-10 from a culture of 1 million monocytes.

Preferred embodiments include methods wherein said monocyte is pretreated with valproic acid at a sufficient concentration and for a time period to stimulate production of at least 10 pg/ml of interleukin-10 from a culture of 1 million monocytes.

Preferred embodiments include methods wherein said monocyte is pretreated with valproic acid at a sufficient concentration and for a time period to stimulate production of at least 20 pg/ml of interleukin-10 from a culture of 1 million monocytes.

Preferred embodiments include methods wherein said monocyte is pretreated with valproic acid at a sufficient concentration and for a time period to stimulate production of at least 40 pg/ml of interleukin-10 from a culture of 1 million monocytes.

Preferred embodiments include methods wherein said immature dendritic cell expresses interleukin-10.

Preferred embodiments include methods wherein said immature dendritic cell expresses interleukin-4.

Preferred embodiments include methods wherein said immature dendritic cell expresses interleukin-13.

Preferred embodiments include methods wherein said immature dendritic cell expresses interleukin-20.

Preferred embodiments include methods wherein said immature dendritic cell expresses TGF-beta.

Preferred embodiments include methods wherein said immature dendritic cell expresses interleukin-20.

Preferred embodiments include methods wherein said immature dendritic cell expresses interleukin-22.

Preferred embodiments include methods wherein said immature dendritic cell expresses interleukin-35.

Preferred embodiments include methods wherein said immature dendritic cell expresses interleukin-37.

Preferred embodiments include methods wherein said immature dendritic cell expresses interleukin-38.

Preferred embodiments include methods wherein said immature dendritic cell expresses TGF-beta.

Preferred embodiments include methods wherein said immature dendritic cell expresses endoglin.

Preferred embodiments include methods wherein said immature dendritic cell expresses VEGF.

Preferred embodiments include methods wherein said immature dendritic cell expresses HLA-G.

Preferred embodiments include methods wherein said immature dendritic cell expresses interleukin-12 p40 homodimer.

Preferred embodiments include methods wherein said immature dendritic cell possesses increased phagocytic activity as compared to a mature dendritic cell.

Preferred embodiments include methods wherein said immature dendritic cell possesses increased migratory activity towards chemotactic gradients as compared to a mature dendritic cell.

Preferred embodiments include methods wherein said chemotactic gradient is comprised of the chemokine IL-8.

Preferred embodiments include methods wherein said chemotactic gradient is comprised of the chemokine MIP-1 alpha.

Preferred embodiments include methods wherein said chemotactic gradient is comprised of the chemokine MIP-1 beta.

Preferred embodiments include methods wherein said chemotactic gradient is comprised of the chemokine CXCL12.

Preferred embodiments include methods wherein said chemotactic gradient is comprised of the chemokine MCP-1.

Preferred embodiments include methods wherein said chemotactic gradient is comprised of the chemokine TNF-alpha.

Preferred embodiments include methods wherein said chemotactic gradient is comprised of the chemokine lymphotoxin.

Preferred embodiments include methods wherein said chemotactic gradient is comprised of the chemokine hyaluronic acid degradation products.

Preferred embodiments include methods wherein said chemotactic gradient is comprised of the chemokine HMGB1.

Preferred embodiments include methods wherein said immature dendritic cell possesses decreased HLA II compared to a mature dendritic cell.

Preferred embodiments include methods wherein said immature dendritic cell possesses decreased HLA I compared to a mature dendritic cell.

Preferred embodiments include methods wherein said immature dendritic cell possesses decreased CD1a compared to a mature dendritic cell.

Preferred embodiments include methods wherein said immature dendritic cell possesses decreased CD40 compared to a mature dendritic cell.

Preferred embodiments include methods wherein said immature dendritic cell possesses decreased CD80 compared to a mature dendritic cell.

Preferred embodiments include methods wherein said immature dendritic cell possesses decreased CD86 compared to a mature dendritic cell.

Preferred embodiments include methods wherein said immature dendritic cell possesses decreased interleukin-15 receptor compared to a mature dendritic cell.

Preferred embodiments include methods wherein said immature dendritic cell possesses decreased interferon gamma compared to a mature dendritic cell.

Preferred embodiments include methods wherein said immature dendritic cell possesses decreased interleukin-18 receptor compared to a mature dendritic cell.

Preferred embodiments include methods wherein said immature dendritic cell possesses decreased progesterone receptor compared to a mature dendritic cell.

Preferred embodiments include methods wherein said immature dendritic cell possesses decreased c-kit compared to a mature dendritic cell.

Preferred embodiments include methods wherein said immature dendritic cell is capable of inducing generation of T regulatory cells upon culture with allogeneic naïve T cells.

Preferred embodiments include methods wherein said immature dendritic cells upregulate expression of AIRE upon contact with said allogeneic naïve T cells.

Preferred embodiments include methods wherein said dendritic cells upregulate expression of AIRE in an interleukin-10 dependent manner.

Preferred embodiments include methods wherein said dendritic cells upregulate expression of AIRE in a TGF-beta dependent manner.

Preferred embodiments include methods wherein said dendritic cells upregulate expression of AIRE in a soluble HLA-G dependent manner.

Preferred embodiments include methods wherein said dendritic cells upregulate expression of AIRE in an endoglin dependent manner.

Preferred embodiments include methods wherein said dendritic cells upregulate expression of AIRE in an FGF-1 dependent manner.

Preferred embodiments include methods wherein said dendritic cells upregulate expression of AIRE in an FGF-2 dependent manner.

Preferred embodiments include methods wherein said dendritic cells upregulate expression of AIRE in an FGF-5 dependent manner.

Preferred embodiments include methods wherein said dendritic cells upregulate expression of AIRE in a NOTCH dependent manner.

Preferred embodiments include methods wherein said myeloid lineage cell is an immature neutrophil.

Preferred embodiments include methods wherein said immature neutrophil is a neutrophil progenitor.

Preferred embodiments include methods wherein said immature neutrophil is capable of differentiating along the neutrophilic lineage or the monocytic lineage.

Preferred embodiments include methods wherein said immature neutrophil expresses PU1.1.

Preferred embodiments include methods wherein said immature neutrophil expresses G-CSF receptor.

Preferred embodiments include methods wherein said immature neutrophil expresses stem cell factor receptor.

Preferred embodiments include methods wherein said immature neutrophil expresses c-met.

Preferred embodiments include methods wherein said immature neutrophil expresses M-CSF receptor.

Preferred embodiments include methods wherein said immature neutrophil expresses GM-CSF receptor.

Preferred embodiments include methods wherein said immature neutrophil produces interleukin-10 upon stimulation with a TLR-4 agonist.

Preferred embodiments include methods wherein said TLR-4 agonist is beta glucan.

Preferred embodiments include methods wherein said TLR-4 agonist is hyaluronic acid degradation products.

Preferred embodiments include methods wherein said TLR-4 agonist is HMGB1.

Preferred embodiments include methods wherein said TLR-4 agonist is histones.

Preferred embodiments include methods wherein said myeloid lineage cell is a myeloid suppressor cell.

Preferred embodiments include methods wherein said myeloid suppressor cell is capable of producing Reptimed.

Preferred embodiments include methods wherein said myeloid suppressor cell is capable of differentiating into monocytes when treated with All Trans Retinoic Acid.

Preferred embodiments include methods wherein said myeloid suppressor cell is capable of differentiating into monocytes when treated with vitamin D3.

Preferred embodiments include methods wherein said myeloid suppressor cell is capable of differentiating into monocytes when treated with M-CSF.

Preferred embodiments include methods wherein said myeloid suppressor cell is capable of differentiating into monocytes when treated with GM-CSF.

Preferred embodiments include methods wherein said myeloid suppressor cell inhibit T cell proliferation by production of nitric oxide.

Preferred embodiments include methods wherein said myeloid suppressor cell inhibit T cell proliferation by production of reactive oxygen species.

Preferred embodiments include methods wherein said myeloid suppressor cell inhibit T cell proliferation by production of superoxide.

Preferred embodiments include methods wherein said myeloid suppressor cell inhibit T cell proliferation by production of hydrogen peroxide.

Preferred embodiments include methods wherein said myeloid suppressor cell inhibit T cell proliferation by production of arginase.

Preferred embodiments include methods wherein said myeloid suppressor cell inhibit T cell proliferation by production of interleukin-10.

Preferred embodiments include methods wherein said myeloid suppressor cell inhibit T cell proliferation by production of soluble PD-L1.

Preferred embodiments include methods wherein said myeloid suppressor cell inhibit T cell proliferation by production of soluble VISTA.

Preferred embodiments include methods wherein said myeloid suppressor cell inhibit T cell proliferation by production of LAG-3.

Preferred embodiments include methods wherein said myeloid suppressor cell inhibit T cell proliferation by production of TIM3.

Preferred embodiments include methods wherein said myeloid suppressor cell inhibit T cell proliferation by production of PGE2.

Preferred embodiments include methods wherein said myeloid suppressor cell inhibit T cell proliferation by production of secreted vimentin.

Preferred embodiments include methods wherein said myeloid suppressor cell inhibit T cell proliferation by production of secreted calreticulin.

Preferred embodiments include methods wherein said T regulatory cell is administered together with a mesenchymal cell differentiated to the oligodendrocyte linage.

Preferred embodiments include methods wherein said T regulatory cell is administered together with a pluripotent stem cell differentiated to the oligodendrocyte linage.

Preferred embodiments include methods wherein said pluripotent stem cell is generated by transfecting a somatic cell with cytoplasm from an oocyte.

Preferred embodiments include methods wherein said cytoplasm is transfected by use of electroporation.

Preferred embodiments include methods wherein said cytoplasm is transfected by use of cell fusion.

Preferred embodiments include methods wherein said cytoplasm is transfected by use of streptolysin O to generate transient holes in the cytoplasm of said recipient cell.

Preferred embodiments include methods wherein said cytoplasm is transfected by use of cell penetrating peptides to generate transient holes in the cytoplasm of said recipient cell.

Preferred embodiments include methods wherein said pluripotent stem cell is an induced pluripotent stem cell.

Preferred embodiments include methods wherein said induced pluripotent stem cell is created by transfection of cells with pluripotency inducing factors.

Preferred embodiments include methods wherein said pluripotency inducing factor is OCT4.

Preferred embodiments include methods wherein said pluripotency inducing factor is PIM-1.

Preferred embodiments include methods wherein said pluripotency inducing factor is NANOG.

Preferred embodiments include methods wherein said pluripotency inducing factor is c-met.

Preferred embodiments include methods wherein said pluripotency inducing factor is hTERT.

Preferred embodiments include methods wherein said pluripotency inducing factor is OCT4.

Preferred embodiments include methods wherein said pluripotency inducing factor is KLF4.

Preferred embodiments include methods wherein said pluripotency inducing factor is RAS.

Preferred embodiments include methods wherein said pluripotency inducing factor is NOTCH.

Preferred embodiments include methods wherein said pluripotency inducing factor is BMP2.

Preferred embodiments include methods wherein said pluripotency inducing factor is BMP4.

Preferred embodiments include methods wherein said pluripotency inducing factor is AIRE.

Preferred embodiments include methods of treating spinal cord injury comprising administration of T regulatory cells in the area of injury using a localization means.

Preferred embodiments include methods wherein said localization means is a decellularized tissue scaffold.

Preferred embodiments include methods wherein said decellularized tissue scaffold is derived from placental chorionic tissue.

Preferred embodiments include methods wherein said decellularized tissue scaffold is derived from placental tissue.

Preferred embodiments include methods wherein said decellularized tissue scaffold is derived from Wharton's Jelly.

Preferred embodiments include methods wherein said decellularized tissue scaffold is derived from subintestinal submucosa.

Preferred embodiments include methods wherein said decellularized tissue scaffold is derived from omental tissue.

Preferred embodiments include methods wherein said decellularized tissue scaffold is derived from lymphatic tissue.

Preferred embodiments include methods wherein said decellularized tissue scaffold is derived from fallopian tube.

Preferred embodiments include methods wherein said decellularized tissue scaffold is derived from cadaveric spinal cord tissue.

Preferred embodiments include methods wherein said decellularization is achieved by treatment with an ionic detergent.

Preferred embodiments include methods wherein said ionic detergent is sodium dodecyl sulfate.

Preferred embodiments include methods wherein said ionic detergent is sodium deoxycholate.

Preferred embodiments include methods wherein said ionic detergent is sodium lauryl ester sulfate.

Preferred embodiments include methods wherein said ionic detergent is sodium lauryl sulfate.

Preferred embodiments include methods wherein said ionic detergent is potassium laurate.

Preferred embodiments include methods wherein said decellularization is achieved by treatment with a nonionic detergent.

Preferred embodiments include methods wherein said nonionic detergent is Triton X-100.

Preferred embodiments include methods wherein said nonionic detergent is Tween 20.

Preferred embodiments include methods wherein said nonionic detergent is Tween 80.

Preferred embodiments include methods wherein said decellularization is achieved by treatment with a zwitterionic detergent.

Preferred embodiments include methods wherein said zwitterionic detergent is CHAPS.

Preferred embodiments include methods wherein said zwitterionic detergent is SB10.

Preferred embodiments include methods wherein said zwitterionic detergent is SB16.

Preferred embodiments include methods wherein said decellularization is achieved by treatment with a solvent.

Preferred embodiments include methods wherein said solvent is an alcohol.

Preferred embodiments include methods wherein said alcohol is methanol or ethanol.

Preferred embodiments include methods wherein said alcohol is selected from a group comprising of: a) isopropyl alcohol; b) acetone; c) tri(n)butyl phosphate; d) urea; and e) dimethyl ester.

Preferred embodiments include methods wherein a histone deacetylase inhibitor is administered locally and/or systemically to enhance reparative activity of said T regulatory cell.

Preferred embodiments include methods wherein said histone deacetylase inhibitor is CXD101.

Preferred embodiments include methods wherein said histone deacetylase inhibitor is HDAC10-IN-1.

Preferred embodiments include methods wherein said histone deacetylase inhibitor is Tubastatin A TFA.

Preferred embodiments include methods wherein said histone deacetylase inhibitor is ACY-775.

Preferred embodiments include methods wherein said histone deacetylase inhibitor is Panobinostat.

Preferred embodiments include methods wherein said histone deacetylase inhibitor is Trichostatin A.

Preferred embodiments include methods wherein said histone deacetylase inhibitor is Vorinostat.

Preferred embodiments include methods wherein said histone deacetylase inhibitor is Entinostat.

Preferred embodiments include methods wherein said histone deacetylase inhibitor is BML-210.

Preferred embodiments include methods wherein said histone deacetylase inhibitor is Abexinostat.

Preferred embodiments include methods wherein said histone deacetylase inhibitor is Dacinostat.

Preferred embodiments include methods wherein said histone deacetylase inhibitor is Quisinostat.

Preferred embodiments include methods wherein said histone deacetylase inhibitor is Mocetinostat.

Preferred embodiments include methods wherein said histone deacetylase inhibitor is Valproic Acid.

Preferred embodiments include methods wherein said histone deacetylase inhibitor is CUDC-101

Preferred embodiments include methods wherein said histone deacetylase inhibitor is GSK3117391.

Preferred embodiments include methods wherein said histone deacetylase inhibitor is Droxinostat.

Preferred embodiments include methods wherein said histone deacetylase inhibitor is MC1568.

Preferred embodiments include methods wherein said histone deacetylase inhibitor is Pracinostat.

Preferred embodiments include methods wherein said histone deacetylase inhibitor is Divalproex Sodium.

Preferred embodiments include methods wherein said histone deacetylase inhibitor is Diferuloylmethane.

Preferred embodiments include methods wherein said histone deacetylase inhibitor is Sodium butyrate.

Preferred embodiments include methods wherein said histone deacetylase inhibitor is PCI-34051.

Preferred embodiments include methods wherein said histone deacetylase inhibitor is SR-4370.

Preferred embodiments include methods wherein said histone deacetylase inhibitor is Givinostat.

Preferred embodiments include methods wherein said histone deacetylase inhibitor is Tubacin.

Preferred embodiments include methods wherein said histone deacetylase inhibitor is AR-42.

Preferred embodiments include methods wherein said histone deacetylase inhibitor is (−)-Parthenolide.

Preferred embodiments include methods wherein said histone deacetylase inhibitor is Tubastatin A HCl.

Preferred embodiments include methods wherein said histone deacetylase inhibitor is Resminostat.

Preferred embodiments include methods wherein said histone deacetylase inhibitor is CUDC-907.

Preferred embodiments include methods wherein said histone deacetylase inhibitor is M344.

Preferred embodiments include methods wherein said histone deacetylase inhibitor is Tacedinaline.

Preferred embodiments include methods wherein said histone deacetylase inhibitor is Romidepsin.

Preferred embodiments include methods wherein said histone deacetylase inhibitor is 4-Phenylbutyric acid.

Preferred embodiments include methods wherein said histone deacetylase inhibitor is Sinapinic acid.

Preferred embodiments include methods wherein said histone deacetylase inhibitor is Sulforaphane.

Preferred embodiments include methods wherein said histone deacetylase inhibitor is UF010.

Preferred embodiments include methods wherein said histone deacetylase inhibitor is Suberohydroxamic acid.

Preferred embodiments include methods wherein said histone deacetylase inhibitor is NKL 22.

Preferred embodiments include methods wherein said histone deacetylase inhibitor is ITSA-1.

Preferred embodiments include methods wherein said histone deacetylase inhibitor is KA2507.

Preferred embodiments include methods wherein said histone deacetylase inhibitor is Isoguanosine.

Preferred embodiments include methods wherein said histone deacetylase inhibitor is Raddeanin A.

Preferred embodiments include methods wherein said histone deacetylase inhibitor is BRD3308.

Preferred embodiments include methods wherein said histone deacetylase inhibitor is TH34.

Preferred embodiments include methods wherein said histone deacetylase inhibitor is Tinostamustine.

Preferred embodiments include methods wherein a mesenchymal stem cell population is administered to the area of injury.

Preferred embodiments include methods wherein said mesenchymal stem cell is transfected with a growth factor.

Preferred embodiments include methods wherein said growth factor is HGF-1.

Preferred embodiments include methods wherein said growth factor is FGF-1.

Preferred embodiments include methods wherein said growth factor is EGF-1.

Preferred embodiments include methods wherein said growth factor is angiopoietin.

Preferred embodiments include methods wherein said growth factor is placental growth factor.

Preferred embodiments include methods wherein said growth factor is adiponectin.

Preferred embodiments include methods wherein said growth factor is vasoactive intestinal peptide precursor.

Preferred embodiments include methods wherein said growth factor is endoglin.

Preferred embodiments include methods wherein said growth factor is myostatin.

Preferred embodiments include methods wherein said growth factor is TGF-beta.

Preferred embodiments include methods wherein said growth factor is vascular endothelial growth factor.

Preferred embodiments include methods wherein said growth factor is GDF-11.

Preferred embodiments include methods wherein said growth factor is GDF-15.

Preferred embodiments include methods wherein said growth factor is hyaluronic acid synthease.

Preferred embodiments include methods wherein said growth factor is interleukin-33.

Preferred embodiments include methods wherein said growth factor is osteosarcoma-derived growth factor.

Preferred embodiments include methods wherein said growth factor is midkine.

Preferred embodiments include methods wherein said growth factor is PDGF-BB.

Preferred embodiments include methods wherein said growth factor is IGF-1.

Preferred embodiments include methods wherein said growth factor is nerve growth factor.

Preferred embodiments include methods wherein said growth factor is brain derived neurotrophic factor.

Preferred embodiments include methods wherein said growth factor is apelin.

Preferred embodiments include methods wherein said T regulatory cell is an effector T regulatory cell.

Preferred embodiments include methods wherein said T regulatory cell is a memory T regulatory cell.

Preferred embodiments include methods wherein said T regulatory cell is a chimeric antigen receptor T regulatory cell.

Preferred embodiments include methods wherein said chimeric antigen receptor T regulatory cell is activated by injury associated antigens.

Preferred embodiments include methods wherein said injury associated antigen is thrombin.

Preferred embodiments include methods wherein said injury associated antigen is chondroitin sulfphate.

Preferred embodiments include methods wherein said injury associated antigen is NOGO.

Preferred embodiments include methods wherein said injury associated antigen is calreticulin.

Preferred embodiments include methods wherein said injury associated antigen is a member of the heat shock protein.

Preferred embodiments include methods wherein said injury associated antigen is vimentin.

Preferred embodiments include methods wherein said injury associated antigen is epidermal growth factor receptor.

Preferred embodiments include methods wherein said injury associated antigen is interleukin-10 receptor.

Preferred embodiments include methods wherein said injury associated antigen is toll like receptor 4.

Preferred embodiments include methods wherein said injury associated antigen is interleukin-4 receptor.

Preferred embodiments include methods wherein said injury associated antigen is interleukin-12 receptor.

Preferred embodiments include methods wherein said injury associated antigen is interleukin-15 receptor.

Preferred embodiments include methods wherein said injury associated antigen is interleukin-17 receptor.

Preferred embodiments include methods wherein said injury associated antigen is interleukin-18 receptor.

Preferred embodiments include methods wherein said injury associated antigen is interleukin-27 receptor.

Preferred embodiments include methods wherein said injury associated antigen is interleukin-33 receptor.

Preferred embodiments include methods wherein said injury associated antigen is toll like receptor 5.

Preferred embodiments include methods wherein said injury associated antigen is toll like receptor 7.

Preferred embodiments include methods wherein said injury associated antigen is toll like receptor 9.

Preferred embodiments include methods wherein said injury associated antigen is TNF-alpha receptor p55.

Preferred embodiments include methods wherein said injury associated antigen is TNF-alpha receptor p75.

Preferred embodiments include methods wherein said mesenchymal stem cell is pretreated with one or more stressing agents to enhance therapeutic activity of stem mesenchymal stem cell.

Preferred embodiments include methods wherein said stressing agent is cobalt chloride.

Preferred embodiments include methods wherein said cobalt chloride is added to said mesenchymal stem cells to allow production of 10 ng/ml or more of VEGF from a culture of 1 million mesenchymal stem cells.

Preferred embodiments include methods wherein said cobalt chloride is added to said mesenchymal stem cells to allow production of 50 ng/ml or more of VEGF from a culture of 1 million mesenchymal stem cells.

Preferred embodiments include methods wherein said cobalt chloride is added to said mesenchymal stem cells to allow production of 100 ng/ml or more of VEGF from a culture of 1 million mesenchymal stem cells.

Preferred embodiments include methods wherein said cobalt chloride is added to said mesenchymal stem cells to allow production of 50 pg/ml or more of EGF from a culture of 1 million mesenchymal stem cells.

Preferred embodiments include methods wherein said cobalt chloride is added to said mesenchymal stem cells to allow production of 100 pg/ml or more of EGF from a culture of 1 million mesenchymal stem cells.

Preferred embodiments include methods wherein said cobalt chloride is added to said mesenchymal stem cells to allow production of 200 pg/ml or more of EGF from a culture of 1 million mesenchymal stem cells.

Preferred embodiments include methods wherein said cobalt chloride is added to said mesenchymal stem cells to allow production of 500 pg/ml or more of BDNF from a culture of 1 million mesenchymal stem cells.

Preferred embodiments include methods wherein said cobalt chloride is added to said mesenchymal stem cells to allow production of 1000 pg/ml or more of BDNF from a culture of 1 million mesenchymal stem cells.

Preferred embodiments include methods wherein said cobalt chloride is added to said mesenchymal stem cells to allow production of 2000 pg/ml or more of BDNF from a culture of 1 million mesenchymal stem cells.

Preferred embodiments include methods wherein said cobalt chloride is added to said mesenchymal stem cells to allow production of 500 pg/ml or more of NGF from a culture of 1 million mesenchymal stem cells.

Preferred embodiments include methods wherein said cobalt chloride is added to said mesenchymal stem cells to allow production of 1000 pg/ml or more of NGF from a culture of 1 million mesenchymal stem cells.

Preferred embodiments include methods wherein said cobalt chloride is added to said mesenchymal stem cells to allow production of 2000 pg/ml or more of NGF from a culture of 1 million mesenchymal stem cells.

Preferred embodiments include methods wherein said cobalt chloride is added to said mesenchymal stem cells to allow production of 500 pg/ml or more of CTNF from a culture of 1 million mesenchymal stem cells.

Preferred embodiments include methods wherein said cobalt chloride is added to said mesenchymal stem cells to allow production of 1000 pg/ml or more of CTNF from a culture of 1 million mesenchymal stem cells.

Preferred embodiments include methods wherein said cobalt chloride is added to said mesenchymal stem cells to allow production of 2000 pg/ml or more of CTNF from a culture of 1 million mesenchymal stem cells.

Preferred embodiments include methods wherein said stressing agent is interferon gamma.

Preferred embodiments include methods wherein said interferon gamma is added to said mesenchymal stem cells to allow production of 10 ng/ml or more of VEGF from a culture of 1 million mesenchymal stem cells.

Preferred embodiments include methods wherein said interferon gamma is added to said mesenchymal stem cells to allow production of 50 ng/ml or more of VEGF from a culture of 1 million mesenchymal stem cells.

Preferred embodiments include methods wherein said interferon gamma is added to said mesenchymal stem cells to allow production of 100 ng/ml or more of VEGF from a culture of 1 million mesenchymal stem cells.

Preferred embodiments include methods wherein said interferon gamma is added to said mesenchymal stem cells to allow production of 50 pg/ml or more of EGF from a culture of 1 million mesenchymal stem cells.

Preferred embodiments include methods wherein said interferon gamma is added to said mesenchymal stem cells to allow production of 100 pg/ml or more of EGF from a culture of 1 million mesenchymal stem cells.

Preferred embodiments include methods wherein said interferon gamma is added to said mesenchymal stem cells to allow production of 200 pg/ml or more of EGF from a culture of 1 million mesenchymal stem cells.

Preferred embodiments include methods wherein said interferon gamma is added to said mesenchymal stem cells to allow production of 500 pg/ml or more of BDNF from a culture of 1 million mesenchymal stem cells.

Preferred embodiments include methods wherein said interferon gamma is added to said mesenchymal stem cells to allow production of 1000 pg/ml or more of BDNF from a culture of 1 million mesenchymal stem cells.

Preferred embodiments include methods wherein said interferon gamma is added to said mesenchymal stem cells to allow production of 2000 pg/ml or more of BDNF from a culture of 1 million mesenchymal stem cells.

Preferred embodiments include methods wherein said interferon gamma is added to said mesenchymal stem cells to allow production of 500 pg/ml or more of NGF from a culture of 1 million mesenchymal stem cells.

Preferred embodiments include methods wherein said interferon gamma is added to said mesenchymal stem cells to allow production of 1000 pg/ml or more of NGF from a culture of 1 million mesenchymal stem cells.

Preferred embodiments include methods wherein said interferon gamma is added to said mesenchymal stem cells to allow production of 2000 pg/ml or more of NGF from a culture of 1 million mesenchymal stem cells.

Preferred embodiments include methods wherein said interferon gamma is added to said mesenchymal stem cells to allow production of 500 pg/ml or more of CTNF from a culture of 1 million mesenchymal stem cells.

Preferred embodiments include methods wherein said interferon gamma is added to said mesenchymal stem cells to allow production of 1000 pg/ml or more of CTNF from a culture of 1 million mesenchymal stem cells.

Preferred embodiments include methods wherein said interferon gamma is added to said mesenchymal stem cells to allow production of 2000 pg/ml or more of CTNF from a culture of 1 million mesenchymal stem cells.

Preferred embodiments include methods wherein said stressing agent is TNF-alpha.

Preferred embodiments include methods wherein said TNF-alpha is added to said mesenchymal stem cells to allow production of 10 ng/ml or more of VEGF from a culture of 1 million mesenchymal stem cells.

Preferred embodiments include methods wherein said TNF-alpha is added to said mesenchymal stem cells to allow production of 50 ng/ml or more of VEGF from a culture of 1 million mesenchymal stem cells.

Preferred embodiments include methods wherein said TNF-alpha is added to said mesenchymal stem cells to allow production of 100 ng/ml or more of VEGF from a culture of 1 million mesenchymal stem cells.

Preferred embodiments include methods wherein said TNF-alpha is added to said mesenchymal stem cells to allow production of 50 pg/ml or more of EGF from a culture of 1 million mesenchymal stem cells.

Preferred embodiments include methods wherein said TNF-alpha is added to said mesenchymal stem cells to allow production of 100 pg/ml or more of EGF from a culture of 1 million mesenchymal stem cells.

Preferred embodiments include methods wherein said TNF-alpha is added to said mesenchymal stem cells to allow production of 200 pg/ml or more of EGF from a culture of 1 million mesenchymal stem cells.

Preferred embodiments include methods wherein said TNF-alpha is added to said mesenchymal stem cells to allow production of 500 pg/ml or more of BDNF from a culture of 1 million mesenchymal stem cells.

Preferred embodiments include methods wherein said TNF-alpha is added to said mesenchymal stem cells to allow production of 1000 pg/ml or more of BDNF from a culture of 1 million mesenchymal stem cells.

Preferred embodiments include methods wherein said TNF-alpha is added to said mesenchymal stem cells to allow production of 2000 pg/ml or more of BDNF from a culture of 1 million mesenchymal stem cells.

Preferred embodiments include methods wherein said TNF-alpha is added to said mesenchymal stem cells to allow production of 500 pg/ml or more of NGF from a culture of 1 million mesenchymal stem cells.

Preferred embodiments include methods wherein said TNF-alpha is added to said mesenchymal stem cells to allow production of 1000 pg/ml or more of NGF from a culture of 1 million mesenchymal stem cells.

Preferred embodiments include methods wherein said TNF-alpha is added to said mesenchymal stem cells to allow production of 2000 pg/ml or more of NGF from a culture of 1 million mesenchymal stem cells.

Preferred embodiments include methods wherein said TNF-alpha is added to said mesenchymal stem cells to allow production of 500 pg/ml or more of CTNF from a culture of 1 million mesenchymal stem cells.

Preferred embodiments include methods wherein said TNF-alpha is added to said mesenchymal stem cells to allow production of 1000 pg/ml or more of CTNF from a culture of 1 million mesenchymal stem cells.

Preferred embodiments include methods wherein said TNF-alpha is added to said mesenchymal stem cells to allow production of 2000 pg/ml or more of CTNF from a culture of 1 million mesenchymal stem cells.

Preferred embodiments include methods wherein said stressing agent is poly IC.

Preferred embodiments include methods wherein said poly IC is added to said mesenchymal stem cells to allow production of 10 ng/ml or more of VEGF from a culture of 1 million mesenchymal stem cells.

Preferred embodiments include methods wherein said poly IC is added to said mesenchymal stem cells to allow production of 50 ng/ml or more of VEGF from a culture of 1 million mesenchymal stem cells.

Preferred embodiments include methods wherein said poly IC is added to said mesenchymal stem cells to allow production of 100 ng/ml or more of VEGF from a culture of 1 million mesenchymal stem cells.

Preferred embodiments include methods wherein said poly IC is added to said mesenchymal stem cells to allow production of 50 pg/ml or more of EGF from a culture of 1 million mesenchymal stem cells.

Preferred embodiments include methods wherein said poly IC is added to said mesenchymal stem cells to allow production of 100 pg/ml or more of EGF from a culture of 1 million mesenchymal stem cells.

Preferred embodiments include methods wherein said poly IC is added to said mesenchymal stem cells to allow production of 200 pg/ml or more of EGF from a culture of 1 million mesenchymal stem cells.

Preferred embodiments include methods wherein said poly IC is added to said mesenchymal stem cells to allow production of 500 pg/ml or more of BDNF from a culture of 1 million mesenchymal stem cells.

Preferred embodiments include methods wherein said poly IC is added to said mesenchymal stem cells to allow production of 1000 pg/ml or more of BDNF from a culture of 1 million mesenchymal stem cells.

Preferred embodiments include methods wherein said poly IC is added to said mesenchymal stem cells to allow production of 2000 pg/ml or more of BDNF from a culture of 1 million mesenchymal stem cells.

Preferred embodiments include methods wherein said poly IC is added to said mesenchymal stem cells to allow production of 500 pg/ml or more of NGF from a culture of 1 million mesenchymal stem cells.

Preferred embodiments include methods wherein said poly IC is added to said mesenchymal stem cells to allow production of 1000 pg/ml or more of NGF from a culture of 1 million mesenchymal stem cells.

Preferred embodiments include methods wherein said poly IC is added to said mesenchymal stem cells to allow production of 2000 pg/ml or more of NGF from a culture of 1 million mesenchymal stem cells.

Preferred embodiments include methods wherein said poly IC is added to said mesenchymal stem cells to allow production of 500 pg/ml or more of CTNF from a culture of 1 million mesenchymal stem cells.

Preferred embodiments include methods wherein said poly IC is added to said mesenchymal stem cells to allow production of 1000 pg/ml or more of CTNF from a culture of 1 million mesenchymal stem cells.

Preferred embodiments include methods wherein said poly IC is added to said mesenchymal stem cells to allow production of 2000 pg/ml or more of CTNF from a culture of 1 million mesenchymal stem cells.

Preferred embodiments include methods wherein said mesenchymal stem cell is cultured with allogeneic stem cells before administration.

Preferred embodiments include methods wherein said allogeneic stem cells are inducible pluripotent stem cells.

Preferred embodiments include methods wherein said mesenchymal stem cells are derived from umbilical cord tissue.

Preferred embodiments include methods wherein said mesenchymal stem cells are derived from umbilical cord tissue.

Preferred embodiments include methods wherein said mesenchymal stem cells are derived from umbilical cord blood.

Preferred embodiments include methods wherein said mesenchymal stem cells are derived from peripheral blood.

Preferred embodiments include methods wherein said mesenchymal stem cells are derived from Wharton's jelly.

Preferred embodiments include methods wherein said mesenchymal stem cells are derived from bone marrow.

Preferred embodiments include methods wherein said mesenchymal stem cells are derived from adipose tissue.

Preferred embodiments include methods wherein said mesenchymal stem cells are derived from endometrium.

Preferred embodiments include methods wherein said mesenchymal stem cells are administered together with one or more angiogenic agents.

Preferred embodiments include methods wherein said angiogenic agent is activin A.

Preferred embodiments include methods wherein said angiogenic agent is adrenomedullin.

Preferred embodiments include methods wherein said angiogenic agent is ALK1.

Preferred embodiments include methods wherein said angiogenic agent is ALK5.

Preferred embodiments include methods wherein said angiogenic agent is ANF.

Preferred embodiments include methods wherein said angiogenic agent is angiogenin.

Preferred embodiments include methods wherein said angiogenic agent is angiopoietin-1.

Preferred embodiments include methods wherein said angiogenic agent is angiopoietin-2.

Preferred embodiments include methods wherein said angiogenic agent is angiopoietin-3.

Preferred embodiments include methods wherein said angiogenic agent is angiopoietin-4.

Preferred embodiments include methods wherein said angiogenic agent is endothelin-1.

Preferred embodiments include methods wherein said angiogenic agent is EDG1.

Preferred embodiments include methods wherein said angiogenic agent is ephrin.

Preferred embodiments include methods wherein said angiogenic agent is erythropoietin.

Preferred embodiments include methods wherein said angiogenic agent is beta thymosin 4.

Preferred embodiments include methods wherein said angiogenic agent is PDGF.

Preferred embodiments include methods wherein said angiogenic agent is PDGF-BB.

Preferred embodiments include methods wherein said angiogenic agent is interleukin-8.

Preferred embodiments include methods wherein said angiogenic agent is interleukin-20.

Preferred embodiments include methods wherein said angiogenic agent is interleukin-22.

Preferred embodiments include methods wherein said angiogenic agent is interleukin-38.

Preferred embodiments include methods wherein said angiogenic agent is fibrin fragment E.

Preferred embodiments include methods wherein said angiogenic agent is factor X.

Preferred embodiments include methods wherein said angiogenic agent is HB-EGF.

Preferred embodiments include methods wherein said angiogenic agent is HBNF.

Preferred embodiments include methods wherein said angiogenic agent is KFGF.

Preferred embodiments include methods wherein said angiogenic agent is leukemia inhibitory factor.

Preferred embodiments include methods wherein said angiogenic agent is leiomyoma-derived growth factor.

Preferred embodiments include methods wherein said angiogenic agent is macrophage-derived growth factor.

Preferred embodiments include methods wherein said angiogenic agent is MCP-1.

Preferred embodiments include methods wherein said angiogenic agent is MD-ECI.

Preferred embodiments include methods wherein said angiogenic agent is GC-MAF

Preferred embodiments include methods wherein said angiogenic agent is MECIF.

Preferred embodiments include methods wherein said angiogenic agent is MMP2.

Preferred embodiments include methods wherein said angiogenic agent is MMP3.

Preferred embodiments include methods wherein said angiogenic agent is MMP9.

Preferred embodiments include methods wherein said angiogenic agent is urokinase plasminogen activator.

Preferred embodiments include methods wherein said angiogenic agent is neuropilin.

Preferred embodiments include methods wherein said angiogenic agent is neurothelin.

Preferred embodiments include methods wherein said angiogenic agent is notch.

Preferred embodiments include methods wherein said angiogenic agent is occludin.

Preferred embodiments include methods wherein said angiogenic agent is zona occludins.

Preferred embodiments include methods wherein said angiogenic agent is oncostatin M.

Preferred embodiments include methods wherein said angiogenic agent is PDECGF.

Preferred embodiments include methods wherein said angiogenic agent is placental factor 4.

Preferred embodiments include methods wherein said angiogenic agent is P1GF.

Preferred embodiments include methods wherein said angiogenic agent is VEGF-A.

Preferred embodiments include methods wherein said angiogenic agent is VEGF-B.

Preferred embodiments include methods wherein said angiogenic agent is VEGF-C.

Preferred embodiments include methods wherein said angiogenic agent is VEGF-D.

Preferred embodiments include methods wherein said angiogenic agent is VEGF-E.

Preferred embodiments include methods wherein said angiogenic agent is VEGF-164.

Preferred embodiments include methods wherein said angiogenic agent is VEGF-I.

Preferred embodiments include methods wherein said angiogenic agent is EC-VEGF.

Preferred embodiments include methods wherein said angiogenic agent is transferrin.

Preferred embodiments include methods wherein said angiogenic agent is thrombospondin.

Preferred embodiments include methods wherein said angiogenic agent is urokinase.

Preferred embodiments include methods wherein said angiogenic agent is administered to the area of spinal cord injury prior to administration of T regulatory cells.

Preferred embodiments include methods wherein said angiogenic agent is administered to the area of spinal cord injury concurrent with administration of T regulatory cells.

Preferred embodiments include methods wherein said angiogenic agent is administered to the area of spinal cord injury subsequent to administration of T regulatory cells.

Preferred embodiments include methods wherein said angiogenic agent is administered to the area of spinal cord injury as a protein.

Preferred embodiments include methods wherein said angiogenic agent is administered to the area of spinal cord injury as a protein-mimetic.

Preferred embodiments include methods wherein said angiogenic agent is administered to the area of spinal cord injury as a receptor agonist.

Preferred embodiments include methods wherein said angiogenic agent is administered to the area of spinal cord injury as a DNA plasmid.

Preferred embodiments include methods wherein said angiogenic agent is administered to the area of spinal cord injury as a microRNA.

Preferred embodiments include methods wherein said angiogenic agent is administered to the area of spinal cord injury as mRNA.

Preferred embodiments include methods wherein said angiogenic agent is administered to the area of spinal cord injury as an active peptide.

Preferred embodiments include methods wherein said angiogenic agent is administered to the area of spinal cord injury as a transfected mesenchymal stem cell.

Preferred embodiments include methods wherein said angiogenic agent is administered to the area of spinal cord injury as a transfected monocyte.

Preferred embodiments include methods wherein said angiogenic agent is administered to the area of spinal cord injury as a transfected B cell.

Preferred embodiments include methods wherein said angiogenic agent is administered to the area of spinal cord injury as a transfected B1 cell.

Preferred embodiments include methods wherein said angiogenic agent is administered to the area of spinal cord injury as a transfected neural progenitor cell.

Preferred embodiments include methods wherein said angiogenic agent is administered to the area of spinal cord injury as a transfected radial cell.

Preferred embodiments include methods wherein said angiogenic agent is administered to the area of spinal cord injury as a transfected oligodendrocyte.

Preferred embodiments include methods wherein said angiogenic agent is administered to the area of spinal cord injury as a transfected fibroblast.

Preferred embodiments include methods wherein said angiogenic agent is administered to the area of spinal cord injury as a transfected astrocyte.

Preferred embodiments include methods wherein said T regulatory cells are administered together with a stem cell chemoattracting cytokine in the area of spinal cord injury.

Preferred embodiments include methods wherein said stem cell chemoattracting cytokine is CXCL12.

Preferred embodiments include methods wherein said stem cell chemoattracting cytokine is MIP-1 alpha.

Preferred embodiments include methods wherein said stem cell chemoattracting cytokine is MIP-1 beta.

Preferred embodiments include methods wherein said stem cell chemoattracting cytokine is VEGF.

Preferred embodiments include methods wherein said stem cell chemoattracting cytokine is RANTES.

Preferred embodiments include methods wherein hematopoietic stem cells are administered together with said T regulatory cells.

Preferred embodiments include methods wherein said hematopoietic stem cell expresses CD34.

Preferred embodiments include methods wherein said hematopoietic stem cell expresses c-kit.

Preferred embodiments include methods wherein said hematopoietic stem cell expresses stem cell factor receptor.

Preferred embodiments include methods wherein said hematopoietic stem cell expresses CD127.

Preferred embodiments include methods wherein said hematopoietic stem cell expresses Fas ligand.

Preferred embodiments include methods wherein said hematopoietic stem cell expresses GITR ligand.

Preferred embodiments include methods wherein said hematopoietic stem cell expresses STAT4.

Preferred embodiments include methods wherein said hematopoietic stem cell expresses SCA-1.

Preferred embodiments include methods wherein said hematopoietic stem cell expresses hTERT.

Preferred embodiments include methods wherein said hematopoietic stem cell expresses NANOG.

Preferred embodiments include methods wherein said hematopoietic stem cell expresses interleukin-6 receptor.

Preferred embodiments include methods wherein said hematopoietic stem cell expresses TGF-beta receptor.

Preferred embodiments include methods wherein said hematopoietic stem cell produces autocrine TGF-beta.

Preferred embodiments include methods wherein said hematopoietic stem cell expresses trk75.

Preferred embodiments include methods wherein said hematopoietic stem cell does not express CD38.

Preferred embodiments include methods wherein said hematopoietic stem cell does not express CD16.

Preferred embodiments include methods wherein said hematopoietic stem cell does not express CD14.

Preferred embodiments include methods wherein said hematopoietic stem cell does not express CD45.

Preferred embodiments include methods wherein said hematopoietic stem cell is allogeneic to the recipient.

Preferred embodiments include methods wherein said hematopoietic stem cell is autologous to the recipient.

Preferred embodiments include methods wherein said hematopoietic stem cell is xenogeneic to the recipient.

Preferred embodiments include methods wherein said hematopoietic stem cell is extracted from bone marrow sources.

Preferred embodiments include methods wherein said hematopoietic stem cell is extracted from peripheral blood sources.

Preferred embodiments include methods wherein said hematopoietic stem cell is extracted from mobilized peripheral blood sources.

Preferred embodiments include methods wherein said hematopoietic stem cell is extracted from cord blood sources.

Preferred embodiments include methods wherein said hematopoietic stem cell is extracted from adipose tissue sources.

Preferred embodiments include methods wherein said hematopoietic stem cell is purified based on expression of CD34.

Preferred embodiments include methods wherein said hematopoietic stem cell is purified based on expression of CD133.

Preferred embodiments include methods wherein said hematopoietic stem cell is purified based on expression of SCA-1 expression.

Preferred embodiments include methods wherein said hematopoietic stem cell is treated with a stressor to enhance spinal cord injury regenerative capacity.

Preferred embodiments include methods wherein said stressor is HMGB1 protein administered for a sufficient concentration and sufficient time period to stimulate production of 10 ng/ml of MMP1 from a culture of 1 million CD34 hematopoietic stem cells.

Preferred embodiments include methods wherein said stressor is HMGB1 protein administered for a sufficient concentration and sufficient time period to stimulate production of 20 ng/ml of MMP1 from a culture of 1 million CD34 hematopoietic stem cells.

Preferred embodiments include methods wherein said stressor is HMGB1 protein administered for a sufficient concentration and sufficient time period to stimulate production of 50 ng/ml of MMP1 from a culture of 1 million CD34 hematopoietic stem cells.

Preferred embodiments include methods wherein said stressor is HMGB1 protein administered for a sufficient concentration and sufficient time period to stimulate production of 50 ng/ml of TGF-beta from a culture of 1 million CD34 hematopoietic stem cells.

Preferred embodiments include methods wherein said stressor is HMGB1 protein administered for a sufficient concentration and sufficient time period to stimulate production of 100 ng/ml of TGF-beta from a culture of 1 million CD34 hematopoietic stem cells.

Preferred embodiments include methods wherein said stressor is HMGB1 protein administered for a sufficient concentration and sufficient time period to stimulate production of 200 ng/ml of TGF-beta from a culture of 1 million CD34 hematopoietic stem cells.

Preferred embodiments include methods wherein said stressor is HMGB1 protein administered for a sufficient concentration and sufficient time period to stimulate production of 1 ng/ml of BDNF from a culture of 1 million CD34 hematopoietic stem cells.

Preferred embodiments include methods wherein said stressor is HMGB1 protein administered for a sufficient concentration and sufficient time period to stimulate production of 5 ng/ml of BDNF from a culture of 1 million CD34 hematopoietic stem cells.

Preferred embodiments include methods wherein said stressor is HMGB1 protein administered for a sufficient concentration and sufficient time period to stimulate production of 10 ng/ml of BDNF from a culture of 1 million CD34 hematopoietic stem cells.

Preferred embodiments include methods wherein said stressor is HMGB1 protein administered for a sufficient concentration and sufficient time period to stimulate production of 20 ng/ml of soluble PD-L1 from a culture of 1 million CD34 hematopoietic stem cells.

Preferred embodiments include methods wherein said stressor is HMGB1 protein administered for a sufficient concentration and sufficient time period to stimulate production of 40 ng/ml of soluble PD-L1from a culture of 1 million CD34 hematopoietic stem cells.

Preferred embodiments include methods wherein said stressor is HMGB1 protein administered for a sufficient concentration and sufficient time period to stimulate production of 80 ng/ml of soluble PD-L1from a culture of 1 million CD34 hematopoietic stem cells.

Preferred embodiments include methods wherein said stressor is HMGB1 protein administered for a sufficient concentration and sufficient time period to stimulate production of 2 ng/ml of irisin from a culture of 1 million CD34 hematopoietic stem cells.

Preferred embodiments include methods wherein said stressor is HMGB1 protein administered for a sufficient concentration and sufficient time period to stimulate production of 4 ng/ml of irisin from a culture of 1 million CD34 hematopoietic stem cells.

Preferred embodiments include methods wherein said stressor is HMGB1 protein administered for a sufficient concentration and sufficient time period to stimulate production of 8 ng/ml of irisin from a culture of 1 million CD34 hematopoietic stem cells.

Preferred embodiments include methods wherein said stressor is HMGB1 protein administered for a sufficient concentration and sufficient time period to stimulate production of 100 pg/ml of soluble HLA-G from a culture of 1 million CD34 hematopoietic stem cells.

Preferred embodiments include methods wherein said stressor is HMGB1 protein administered for a sufficient concentration and sufficient time period to stimulate production of 200 pg/ml of soluble HLA-G from a culture of 1 million CD34 hematopoietic stem cells.

Preferred embodiments include methods wherein said stressor is HMGB1 protein administered for a sufficient concentration and sufficient time period to stimulate production of 400 pg/ml of soluble HLA-G from a culture of 1 million CD34 hematopoietic stem cells.

Preferred embodiments include methods wherein said stressor is flagellin protein administered for a sufficient concentration and sufficient time period to stimulate production of 10 ng/ml of MMP1 from a culture of 1 million CD34 hematopoietic stem cells.

Preferred embodiments include methods wherein said stressor is flagellin protein administered for a sufficient concentration and sufficient time period to stimulate production of 20 ng/ml of MMP1 from a culture of 1 million CD34 hematopoietic stem cells.

Preferred embodiments include methods wherein said stressor is flagellin protein administered for a sufficient concentration and sufficient time period to stimulate production of 50 ng/ml of MMP1 from a culture of 1 million CD34 hematopoietic stem cells.

Preferred embodiments include methods wherein said stressor is flagellin protein administered for a sufficient concentration and sufficient time period to stimulate production of 50 ng/ml of TGF-beta from a culture of 1 million CD34 hematopoietic stem cells.

Preferred embodiments include methods wherein said stressor is flagellin protein administered for a sufficient concentration and sufficient time period to stimulate production of 100 ng/ml of TGF-beta from a culture of 1 million CD34 hematopoietic stem cells.

Preferred embodiments include methods wherein said stressor is flagellin protein administered for a sufficient concentration and sufficient time period to stimulate production of 200 ng/ml of TGF-beta from a culture of 1 million CD34 hematopoietic stem cells.

Preferred embodiments include methods wherein said stressor is flagellin protein administered for a sufficient concentration and sufficient time period to stimulate production of 1 ng/ml of BDNF from a culture of 1 million CD34 hematopoietic stem cells.

Preferred embodiments include methods wherein said stressor is flagellin protein administered for a sufficient concentration and sufficient time period to stimulate production of 5 ng/ml of BDNF from a culture of 1 million CD34 hematopoietic stem cells.

Preferred embodiments include methods wherein said stressor is flagellin protein administered for a sufficient concentration and sufficient time period to stimulate production of 10 ng/ml of BDNF from a culture of 1 million CD34 hematopoietic stem cells.

Preferred embodiments include methods wherein said stressor is flagellin protein administered for a sufficient concentration and sufficient time period to stimulate production of 20 ng/ml of soluble PD-L1 from a culture of 1 million CD34 hematopoietic stem cells.

Preferred embodiments include methods wherein said stressor is flagellin protein administered for a sufficient concentration and sufficient time period to stimulate production of 40 ng/ml of soluble PD-L1from a culture of 1 million CD34 hematopoietic stem cells.

Preferred embodiments include methods wherein said stressor is flagellin protein administered for a sufficient concentration and sufficient time period to stimulate production of 80 ng/ml of soluble PD-L1from a culture of 1 million CD34 hematopoietic stem cells.

Preferred embodiments include methods wherein said stressor is flagellin protein administered for a sufficient concentration and sufficient time period to stimulate production of 2 ng/ml of irisin from a culture of 1 million CD34 hematopoietic stem cells.

Preferred embodiments include methods wherein said stressor is flagellin protein administered for a sufficient concentration and sufficient time period to stimulate production of 4 ng/ml of irisin from a culture of 1 million CD34 hematopoietic stem cells.

Preferred embodiments include methods wherein said stressor is flagellin protein administered for a sufficient concentration and sufficient time period to stimulate production of 8 ng/ml of irisin from a culture of 1 million CD34 hematopoietic stem cells.

Preferred embodiments include methods wherein said stressor is flagellin protein administered for a sufficient concentration and sufficient time period to stimulate production of 100 pg/ml of soluble HLA-G from a culture of 1 million CD34 hematopoietic stem cells.

Preferred embodiments include methods wherein said stressor is flagellin protein administered for a sufficient concentration and sufficient time period to stimulate production of 200 pg/ml of soluble HLA-G from a culture of 1 million CD34 hematopoietic stem cells.

Preferred embodiments include methods wherein said stressor is flagellin protein administered for a sufficient concentration and sufficient time period to stimulate production of 400 pg/ml of soluble HLA-G from a culture of 1 million CD34 hematopoietic stem cells.

Preferred embodiments include methods wherein said stressor is imiquimod administered for a sufficient concentration and sufficient time period to stimulate production of 10 ng/ml of MMP1 from a culture of 1 million CD34 hematopoietic stem cells.

Preferred embodiments include methods wherein said stressor is imiquimod administered for a sufficient concentration and sufficient time period to stimulate production of 20 ng/ml of MMP1 from a culture of 1 million CD34 hematopoietic stem cells.

Preferred embodiments include methods wherein said stressor is imiquimod administered for a sufficient concentration and sufficient time period to stimulate production of 50 ng/ml of MMP1 from a culture of 1 million CD34 hematopoietic stem cells.

Preferred embodiments include methods wherein said stressor is imiquimod administered for a sufficient concentration and sufficient time period to stimulate production of 50 ng/ml of TGF-beta from a culture of 1 million CD34 hematopoietic stem cells.

Preferred embodiments include methods wherein said stressor is imiquimod administered for a sufficient concentration and sufficient time period to stimulate production of 100 ng/ml of TGF-beta from a culture of 1 million CD34 hematopoietic stem cells.

Preferred embodiments include methods wherein said stressor is imiquimod administered for a sufficient concentration and sufficient time period to stimulate production of 200 ng/ml of TGF-beta from a culture of 1 million CD34 hematopoietic stem cells.

Preferred embodiments include methods wherein said stressor is imiquimod administered for a sufficient concentration and sufficient time period to stimulate production of 1 ng/ml of BDNF from a culture of 1 million CD34 hematopoietic stem cells.

Preferred embodiments include methods wherein said stressor is imiquimod administered for a sufficient concentration and sufficient time period to stimulate production of 5 ng/ml of BDNF from a culture of 1 million CD34 hematopoietic stem cells.

Preferred embodiments include methods wherein said stressor is imiquimod administered for a sufficient concentration and sufficient time period to stimulate production of 10 ng/ml of BDNF from a culture of 1 million CD34 hematopoietic stem cells.

Preferred embodiments include methods wherein said stressor is imiquimod administered for a sufficient concentration and sufficient time period to stimulate production of 20 ng/ml of soluble PD-L1 from a culture of 1 million CD34 hematopoietic stem cells.

Preferred embodiments include methods wherein said stressor is imiquimod administered for a sufficient concentration and sufficient time period to stimulate production of 40 ng/ml of soluble PD-L1from a culture of 1 million CD34 hematopoietic stem cells.

Preferred embodiments include methods wherein said stressor is imiquimod administered for a sufficient concentration and sufficient time period to stimulate production of 80 ng/ml of soluble PD-L1from a culture of 1 million CD34 hematopoietic stem cells.

Preferred embodiments include methods wherein said stressor is imiquimod administered for a sufficient concentration and sufficient time period to stimulate production of 2 ng/ml of irisin from a culture of 1 million CD34 hematopoietic stem cells.

Preferred embodiments include methods wherein said stressor is imiquimod administered for a sufficient concentration and sufficient time period to stimulate production of 4 ng/ml of irisin from a culture of 1 million CD34 hematopoietic stem cells.

Preferred embodiments include methods wherein said stressor is imiquimod administered for a sufficient concentration and sufficient time period to stimulate production of 8 ng/ml of irisin from a culture of 1 million CD34 hematopoietic stem cells.

Preferred embodiments include methods wherein said stressor is imiquimod administered for a sufficient concentration and sufficient time period to stimulate production of 100 pg/ml of soluble HLA-G from a culture of 1 million CD34 hematopoietic stem cells.

Preferred embodiments include methods wherein said stressor is imiquimod administered for a sufficient concentration and sufficient time period to stimulate production of 200 pg/ml of soluble HLA-G from a culture of 1 million CD34 hematopoietic stem cells.

Preferred embodiments include methods wherein said stressor is imiquimod administered for a sufficient concentration and sufficient time period to stimulate production of 400 pg/ml of soluble HLA-G from a culture of 1 million CD34 hematopoietic stem cells.

Preferred embodiments include methods wherein said stressor is poly IC administered for a sufficient concentration and sufficient time period to stimulate production of 10 ng/ml of MMP1 from a culture of 1 million CD34 hematopoietic stem cells.

Preferred embodiments include methods wherein said stressor is poly IC administered for a sufficient concentration and sufficient time period to stimulate production of 20 ng/ml of MMP1 from a culture of 1 million CD34 hematopoietic stem cells.

Preferred embodiments include methods wherein said stressor is poly IC administered for a sufficient concentration and sufficient time period to stimulate production of 50 ng/ml of MMP1 from a culture of 1 million CD34 hematopoietic stem cells.

Preferred embodiments include methods wherein said stressor is poly IC administered for a sufficient concentration and sufficient time period to stimulate production of 50 ng/ml of TGF-beta from a culture of 1 million CD34 hematopoietic stem cells.

Preferred embodiments include methods wherein said stressor is poly IC administered for a sufficient concentration and sufficient time period to stimulate production of 100 ng/ml of TGF-beta from a culture of 1 million CD34 hematopoietic stem cells.

Preferred embodiments include methods wherein said stressor is poly IC administered for a sufficient concentration and sufficient time period to stimulate production of 200 ng/ml of TGF-beta from a culture of 1 million CD34 hematopoietic stem cells.

Preferred embodiments include methods wherein said stressor is poly IC administered for a sufficient concentration and sufficient time period to stimulate production of 1 ng/ml of BDNF from a culture of 1 million CD34 hematopoietic stem cells.

Preferred embodiments include methods wherein said stressor is poly IC administered for a sufficient concentration and sufficient time period to stimulate production of 5 ng/ml of BDNF from a culture of 1 million CD34 hematopoietic stem cells.

Preferred embodiments include methods wherein said stressor is poly IC administered for a sufficient concentration and sufficient time period to stimulate production of 10 ng/ml of BDNF from a culture of 1 million CD34 hematopoietic stem cells.

Preferred embodiments include methods wherein said stressor is poly IC administered for a sufficient concentration and sufficient time period to stimulate production of 20 ng/ml of soluble PD-L1 from a culture of 1 million CD34 hematopoietic stem cells.

Preferred embodiments include methods wherein said stressor is poly IC administered for a sufficient concentration and sufficient time period to stimulate production of 40 ng/ml of soluble PD-L1 from a culture of 1 million CD34 hematopoietic stem cells.

Preferred embodiments include methods wherein said stressor is poly IC administered for a sufficient concentration and sufficient time period to stimulate production of 80 ng/ml of soluble PD-L1 from a culture of 1 million CD34 hematopoietic stem cells.

Preferred embodiments include methods wherein said stressor is poly IC administered for a sufficient concentration and sufficient time period to stimulate production of 2 ng/ml of irisin from a culture of 1 million CD34 hematopoietic stem cells.

Preferred embodiments include methods wherein said stressor is poly IC administered for a sufficient concentration and sufficient time period to stimulate production of 4 ng/ml of irisin from a culture of 1 million CD34 hematopoietic stem cells.

Preferred embodiments include methods wherein said stressor is poly IC administered for a sufficient concentration and sufficient time period to stimulate production of 8 ng/ml of irisin from a culture of 1 million CD34 hematopoietic stem cells.

Preferred embodiments include methods wherein said stressor is poly IC administered for a sufficient concentration and sufficient time period to stimulate production of 100 pg/ml of soluble HLA-G from a culture of 1 million CD34 hematopoietic stem cells.

Preferred embodiments include methods wherein said stressor is poly IC administered for a sufficient concentration and sufficient time period to stimulate production of 200 pg/ml of soluble HLA-G from a culture of 1 million CD34 hematopoietic stem cells.

Preferred embodiments include methods wherein said stressor is poly IC administered for a sufficient concentration and sufficient time period to stimulate production of 400 pg/ml of soluble HLA-G from a culture of 1 million CD34 hematopoietic stem cells.

Preferred embodiments include methods wherein said stressor is BCG administered for a sufficient concentration and sufficient time period to stimulate production of 10 ng/ml of MMP1 from a culture of 1 million CD34 hematopoietic stem cells.

Preferred embodiments include methods wherein said stressor is BCG administered for a sufficient concentration and sufficient time period to stimulate production of 20 ng/ml of MMP1 from a culture of 1 million CD34 hematopoietic stem cells.

Preferred embodiments include methods wherein said stressor is BCG administered for a sufficient concentration and sufficient time period to stimulate production of 50 ng/ml of MMP1 from a culture of 1 million CD34 hematopoietic stem cells.

Preferred embodiments include methods wherein said stressor is BCG administered for a sufficient concentration and sufficient time period to stimulate production of 50 ng/ml of TGF-beta from a culture of 1 million CD34 hematopoietic stem cells.

Preferred embodiments include methods wherein said stressor is BCG administered for a sufficient concentration and sufficient time period to stimulate production of 100 ng/ml of TGF-beta from a culture of 1 million CD34 hematopoietic stem cells.

Preferred embodiments include methods wherein said stressor is BCG administered for a sufficient concentration and sufficient time period to stimulate production of 200 ng/ml of TGF-beta from a culture of 1 million CD34 hematopoietic stem cells.

Preferred embodiments include methods wherein said stressor is BCG administered for a sufficient concentration and sufficient time period to stimulate production of 1 ng/ml of BDNF from a culture of 1 million CD34 hematopoietic stem cells.

Preferred embodiments include methods wherein said stressor is BCG administered for a sufficient concentration and sufficient time period to stimulate production of 5 ng/ml of BDNF from a culture of 1 million CD34 hematopoietic stem cells.

Preferred embodiments include methods wherein said stressor is BCG administered for a sufficient concentration and sufficient time period to stimulate production of 10 ng/ml of BDNF from a culture of 1 million CD34 hematopoietic stem cells.

Preferred embodiments include methods wherein said stressor is BCG administered for a sufficient concentration and sufficient time period to stimulate production of 20 ng/ml of soluble PD-L1 from a culture of 1 million CD34 hematopoietic stem cells.

Preferred embodiments include methods wherein said stressor is BCG administered for a sufficient concentration and sufficient time period to stimulate production of 40 ng/ml of soluble PD-L1 from a culture of 1 million CD34 hematopoietic stem cells.

Preferred embodiments include methods wherein said stressor is BCG administered for a sufficient concentration and sufficient time period to stimulate production of 80 ng/ml of soluble PD-L1 from a culture of 1 million CD34 hematopoietic stem cells.

Preferred embodiments include methods wherein said stressor is BCG administered for a sufficient concentration and sufficient time period to stimulate production of 2 ng/ml of irisin from a culture of 1 million CD34 hematopoietic stem cells.

Preferred embodiments include methods wherein said stressor is BCG administered for a sufficient concentration and sufficient time period to stimulate production of 4 ng/ml of irisin from a culture of 1 million CD34 hematopoietic stem cells.

Preferred embodiments include methods wherein said stressor is BCG administered for a sufficient concentration and sufficient time period to stimulate production of 8 ng/ml of irisin from a culture of 1 million CD34 hematopoietic stem cells.

Preferred embodiments include methods wherein said stressor is BCG administered for a sufficient concentration and sufficient time period to stimulate production of 100 pg/ml of soluble HLA-G from a culture of 1 million CD34 hematopoietic stem cells.

Preferred embodiments include methods wherein said stressor is BCG administered for a sufficient concentration and sufficient time period to stimulate production of 200 pg/ml of soluble HLA-G from a culture of 1 million CD34 hematopoietic stem cells.

Preferred embodiments include methods wherein said stressor is BCG administered for a sufficient concentration and sufficient time period to stimulate production of 400 pg/ml of soluble HLA-G from a culture of 1 million CD34 hematopoietic stem cells.

Preferred embodiments include methods wherein said patient is administered one or more agents capable of increasing T regulatory cell numbers locally.

Preferred embodiments include methods wherein said patient is administered one or more agents capable of increasing T regulatory cell numbers systemically.

Preferred embodiments include methods wherein said agent is interleukin-2.

Preferred embodiments include methods wherein said agent is interleukin-10.

Preferred embodiments include methods wherein said agent is TGF-beta.

Preferred embodiments include methods wherein said agent is a cell population associated with tolerance induction.

Preferred embodiments include methods wherein said cell associated with tolerance induction is a monocyte treated with interleukin-4 so as to endow said monocyte with ability to produce more arginase than nitric oxide synthase.

Preferred embodiments include methods wherein said cell associated with tolerance induction is a monocyte treated with interleukin-10 so as to endow said monocyte with ability to produce more arginase than nitric oxide synthase.

Preferred embodiments include methods wherein said cell associated with tolerance induction is a monocyte treated with TGF-beta so as to endow said monocyte with ability to produce more arginase than nitric oxide synthase.

Preferred embodiments include methods wherein said cell associated with tolerance induction is a monocyte treated with VEGF so as to endow said monocyte with ability to produce more arginase than nitric oxide synthase.

Preferred embodiments include methods wherein said cell associated with tolerance induction is a monocyte treated with an inhibitor of interferon gamma receptor so as to endow said monocyte with ability to produce more arginase than nitric oxide synthase.

Preferred embodiments include methods wherein said cell associated with tolerance induction is a monocyte transfected with CTLA-4.

Preferred embodiments include methods wherein said cell associated with tolerance induction is a monocyte transfected with PD-1.

Preferred embodiments include methods wherein said cell associated with tolerance induction is a monocyte transfected with VISTA.

Preferred embodiments include methods wherein said cell associated with tolerance induction is a monocyte transfected with B7-H2.

Preferred embodiments include methods wherein said cell associated with tolerance induction is a monocyte transfected with B7-H3.

Preferred embodiments include methods wherein said cell associated with tolerance induction is a monocyte transfected with B7-H4.

Preferred embodiments include methods wherein said cell associated with tolerance induction is a monocyte transfected with PD-L1.

Preferred embodiments include methods wherein said cell associated with tolerance induction is a monocyte transfected with ICOS.

Preferred embodiments include methods wherein said cell associated with tolerance induction is a monocyte transfected with HVEM.

Preferred embodiments include methods wherein said cell associated with tolerance induction is a monocyte transfected with PD-L2.

Preferred embodiments include methods wherein said cell associated with tolerance induction is a monocyte transfected with CD160.

Preferred embodiments include methods wherein said cell associated with tolerance induction is a monocyte transfected with gp49B.

Preferred embodiments include methods wherein said cell associated with tolerance induction is a monocyte transfected with PIR-B.

Preferred embodiments include methods wherein said cell associated with tolerance induction is a monocyte transfected with TIM-1.

Preferred embodiments include methods wherein said cell associated with tolerance induction is a monocyte transfected with TIM-3.

Preferred embodiments include methods wherein said cell associated with tolerance induction is a monocyte transfected with TIM-4.

Preferred embodiments include methods wherein said cell associated with tolerance induction is a monocyte transfected with LAG-3.

Preferred embodiments include methods wherein said cell associated with tolerance induction is a monocyte transfected with GITR.

Preferred embodiments include methods wherein said cell associated with tolerance induction is a monocyte transfected with 4-1BB.

Preferred embodiments include methods wherein said cell associated with tolerance induction is a monocyte transfected with OX-40.

Preferred embodiments include methods wherein said cell associated with tolerance induction is a monocyte transfected with BTLA.

Preferred embodiments include methods wherein said cell associated with tolerance induction is a monocyte transfected with CD47.

Preferred embodiments include methods wherein said cell associated with tolerance induction is a monocyte transfected with CD-48.

Preferred embodiments include methods wherein said cell associated with tolerance induction is a monocyte transfected with CD244.

Preferred embodiments include methods wherein said cell associated with tolerance induction is a monocyte transfected with ILT2.

Preferred embodiments include methods wherein said cell associated with tolerance induction is a monocyte transfected with ILT4.

Preferred embodiments include methods wherein said cell associated with tolerance induction is a monocyte transfected with OX-40.

Preferred embodiments include methods wherein said cell associated with tolerance induction is a monocyte transfected with TIGIT.

Preferred embodiments include methods wherein said cell associated with tolerance induction is a monocyte transfected with A2aR.

Preferred embodiments include methods wherein said cell associated with tolerance induction is a monocyte transfected with interleukin 1 receptor antagonist.

Preferred embodiments include methods wherein said cell associated with tolerance induction is a monocyte transfected with soluble HLA-G.

Preferred embodiments include methods wherein said cell associated with tolerance induction is a monocyte transfected with Fas ligand.

Preferred embodiments include methods wherein said cell associated with tolerance induction is a monocyte transfected with soluble TNF receptor p55.

Preferred embodiments include methods wherein said cell associated with tolerance induction is a monocyte transfected with soluble TNF receptor p75.

Preferred embodiments include methods wherein said cell associated with tolerance induction is a mesenchymal stem cell transfected with CTLA-4.

Preferred embodiments include methods wherein said cell associated with tolerance induction is a mesenchymal stem cell transfected with PD-1.

Preferred embodiments include methods wherein said cell associated with tolerance induction is a mesenchymal stem cell transfected with VISTA.

Preferred embodiments include methods wherein said cell associated with tolerance induction is a mesenchymal stem cell transfected with B7-H2.

Preferred embodiments include methods wherein said cell associated with tolerance induction is a mesenchymal stem cell transfected with B7-H3.

Preferred embodiments include methods wherein said cell associated with tolerance induction is a mesenchymal stem cell transfected with B7-H4.

Preferred embodiments include methods wherein said cell associated with tolerance induction is a mesenchymal stem cell transfected with PD-L1.

Preferred embodiments include methods wherein said cell associated with tolerance induction is a mesenchymal stem cell transfected with ICOS.

Preferred embodiments include methods wherein said cell associated with tolerance induction is a mesenchymal stem cell transfected with HVEM.

Preferred embodiments include methods wherein said cell associated with tolerance induction is a mesenchymal stem cell transfected with PD-L2.

Preferred embodiments include methods wherein said cell associated with tolerance induction is a mesenchymal stem cell transfected with CD160.

Preferred embodiments include methods wherein said cell associated with tolerance induction is a mesenchymal stem cell transfected with gp49B.

Preferred embodiments include methods wherein said cell associated with tolerance induction is a mesenchymal stem cell transfected with PIR-B.

Preferred embodiments include methods wherein said cell associated with tolerance induction is a mesenchymal stem cell transfected with TIM-1.

Preferred embodiments include methods wherein said cell associated with tolerance induction is a mesenchymal stem cell transfected with TIM-3.

Preferred embodiments include methods wherein said cell associated with tolerance induction is a mesenchymal stem cell transfected with TIM-4.

Preferred embodiments include methods wherein said cell associated with tolerance induction is a mesenchymal stem cell transfected with LAG-3.

Preferred embodiments include methods wherein said cell associated with tolerance induction is a mesenchymal stem cell transfected with GITR.

Preferred embodiments include methods wherein said cell associated with tolerance induction is a mesenchymal stem cell transfected with 4-1BB.

Preferred embodiments include methods wherein said cell associated with tolerance induction is a mesenchymal stem cell transfected with OX-40.

Preferred embodiments include methods wherein said cell associated with tolerance induction is a mesenchymal stem cell transfected with BTLA.

Preferred embodiments include methods wherein said cell associated with tolerance induction is a mesenchymal stem cell transfected with CD47.

Preferred embodiments include methods wherein said cell associated with tolerance induction is a mesenchymal stem cell transfected with CD-48.

Preferred embodiments include methods wherein said cell associated with tolerance induction is a mesenchymal stem cell transfected with CD244.

Preferred embodiments include methods wherein said cell associated with tolerance induction is a mesenchymal stem cell transfected with ILT2.

Preferred embodiments include methods wherein said cell associated with tolerance induction is a mesenchymal stem cell transfected with ILT4.

Preferred embodiments include methods wherein said cell associated with tolerance induction is a mesenchymal stem cell transfected with OX-40.

Preferred embodiments include methods wherein said cell associated with tolerance induction is a mesenchymal stem cell transfected with TIGIT.

Preferred embodiments include methods wherein said cell associated with tolerance induction is a mesenchymal stem cell transfected with A2aR.

Preferred embodiments include methods wherein said cell associated with tolerance induction is a mesenchymal stem cell transfected with interleukin 1 receptor antagonist.

Preferred embodiments include methods wherein said cell associated with tolerance induction is a mesenchymal stem cell transfected with soluble HLA-G.

Preferred embodiments include methods wherein said cell associated with tolerance induction is a mesenchymal stem cell transfected with Fas ligand.

Preferred embodiments include methods wherein said cell associated with tolerance induction is a mesenchymal stem cell transfected with soluble TNF receptor p55.

Preferred embodiments include methods wherein said cell associated with tolerance induction is a mesenchymal stem cell transfected with soluble TNF receptor p75.

Preferred embodiments include methods wherein extracorporeal pulsed ultrasound is administered prior to infusion of T regulatory cells.

Preferred embodiments include methods wherein extracorporeal pulsed ultrasound is administered concurrent with infusion of T regulatory cells.

Preferred embodiments include methods wherein extracorporeal pulsed ultrasound is administered subsequent to infusion of T regulatory cells.

Preferred embodiments include methods wherein said T regulatory cells are administered together with oligodendrocyte progenitor cells.

Preferred embodiments include methods wherein said oligodendrocyte progenitor cells are derived from pluripotent stem cells.

Preferred embodiments include methods wherein said oligodendrocyte progenitor cells are derived from an autologous source.

Preferred embodiments include methods wherein said oligodendrocyte progenitor cells are derived from an allogeneic source.

Preferred embodiments include methods wherein said oligodendrocyte progenitor cells are derived from a xenogeneic source.

Preferred embodiments include methods wherein said oligodendrocyte progenitor cells are administered together with a complement inhibitor.

Preferred embodiments include methods wherein said complement inhibitor is anti-C5 antibody.

Preferred embodiments include methods wherein said complement inhibitor is anti-C3 antibody.

Preferred embodiments include methods wherein said complement inhibitor is cobra venom factor.

Preferred embodiments include methods wherein said complement inhibitor is Factor H.

Preferred embodiments include methods wherein said complement inhibitor is CD55.

Preferred embodiments include methods wherein said complement inhibitor is CD37.

Preferred embodiments include methods wherein said complement inhibitor is CD73.

### DETAILED DESCRIPTION OF THE INVENTION

The invention teaches the use of T regulatory cells for the treatment of spinal cord injury. In one embodiment the invention provides the previously unknown ability of T regulatory cells to induce neurogenesis and augment neuronal plasticity following injury. T regulatory cells utilized in the invention may be derived and expanded from natural sources such as peripheral blood, bone marrow, or adipose tissue, or they may be generated from progenitor or stem cell populations. In one embodiment spinal cord injury is treated with T regulatory cells obtained from pluripotent stem cells such as induced pluripotent stem cells, embryonic stem cells, somatic cell nuclear transfer derived stem cells,

The term “administration” or “administer” refers to the direct application of a therapeutically effective amount of the composition of the present invention to a subject, to thereby form the same amount thereof in the body of the subject. Therefore, the term “administer” includes the injection of an active ingredient (neural cells) around a site of lesion, and thus the term is used in the same meaning as the term “inject”.

The term “treatment” refers to: (a) suppressing the development of disease, disorder, or symptom; (b) reducing disease, disorder, or symptom; or (c) curing disease, disorder, or symptom. The composition of the present invention either suppresses the development of, inhibits, or reduces the symptom of spinal cord injury upon transplanting the neural precursor cells to a subject with spinal cord injury. Therefore, the composition of the present invention per se may be a composition for treating spinal cord injury, or the composition of the present invention may be applied as a treatment adjuvant by the administration thereof together with another composition for treating spinal cord injury. Therefore, as used herein, the term “treatment” or “treatment agent” includes a meaning of “treatment aid” or “treatment adjuvant”.

The term “therapeutically effective amount” refers to the content of cells, which is sufficient to provide a therapeutic or prophylactic effect to a subject to which the composition is to be administered, and thus the term has a meaning including “prophylactically effective amount”. As used herein, the term “subject” includes, but is not limited to, human, mouse, rat, guinea pig, dog, cat, horse, cow, pig, monkey, chimpanzee, beaver, or rhesus monkey. Specifically, the subject of the present invention is human.

The term “stem cells” is a generic term for undifferentiated cells before differentiation into respective cells constituting tissues, and the stem cells have an ability to be differentiated into particular cells by particular differentiation stimuli (environment). Unlike cell division-ceased terminally differentiated cells, the stem cells are capable of producing the identical cells through cell division (self-renewal), and have plasticity in differentiation, in which the stem cells are differentiated into particular cells by differentiation inducing stimuli and may be differentiated into other cell types under different environments or by different stimuli. Pluripotent stem cells that proliferate indefinitely in vitro and can be differentiated into various cells derived from all embryonic layers (ectoderm, mesoderm, and endoderm). More specifically, the pluripotent stem cells are embryonic stem cells, induced pluripotent stem cells (iPSCs), embryonic germ cells, or embryonic carcinoma cells. The embryonic stem cells are derived from the inner cell mass (ICM) of the blastocyst, and the embryonic germ cells are derived from primordial germ cells present in 5-10 week-old gonadal ridges. Induced pluripotent stem cells (iPSCs) are one type of pluripotent stem cells artificially derived from non-pluripotent cells (e.g., somatic cells) by inserting a particular gene imparting pluripotency therein. Induced pluripotent stem cells are considered to be the same as pluripotent stem cells (e.g., embryonic stem cells) since the induced pluripotent stem cells have highly similar stem cell gene and protein expression patterns, chromosomal methylation pattern, doubling time, embryoid body formation capacity, teratoma formation capacity, viable chimera formation capacity, hybridizability, and differentiability ability as embryonic stem cells.

The invention provides the use of T regulatory cells, primarily FoxP3 expressing T regulatory cells for treatment of various types of spinal cord injury. The syndrome or condition of discomfort of the subject associated with the spinal injury being treated varies according to the type and level of the injury. In addition to a loss of sensation and motor function below the point of injury, individuals with spinal cord injuries will often experience other complications of spinal cord injury, such as dysfunction of the bowel and bladder, including infections of the bladder, and anal incontinence; sexual dysfunction; loss of breathing, necessitating mechanical ventilators or phrenic nerve pacing; inability or reduced ability to regulate heart rate and/or blood pressure, sweating and hence body temperature; spasticity (increased reflexes and stiffness of the limbs); neuropathic pain; autonomic dysreflexia or abnormal increases in blood pressure, sweating, and other autonomic responses to pain or sensory disturbances; atrophy of muscle; osteoporosis (loss of calcium) and bone degeneration; and gall bladder and renal stones.

Use of T regulatory cells is provided at various concentrations and/or in combinations with other cell types such as monocytes, alternatively activated monocytes, gamma delta T cells, natural killer cells, and B1 cells. In one embodiment, the observable beneficial effect is a complete recovery from the spinal injury. In another embodiment, the observable beneficial effect is an improvement in the pathological conditions of the subject suffering from a spinal injury. In yet another embodiment, the observable beneficial effect is a restoration, completely or partially, of the physical function of the injured spinal cord. Methods are known in the art for determining therapeutically effective doses of T regulatory cells according to the present disclosure. A useful assay for confirming an effective amount (e.g., a therapeutically effective amount) for a predetermined application is to measure the degree of recovery from a target disease, disorder or condition. An amount of T regulatory cells actually transplanted to the area of spinal injury varies in view of many parameters, such as the condition of the subject, the type and severity of the spinal injury, the route of transplantation, e.g., direct or indirect, etc. The amount of T regulatory cells, when applied to the subject suffering from spinal injury, should attain a desired effect, i.e., repairs spinal injury and/or enhances at least partially functional recovery of the injured spinal cord, without significant side effects. The determination of a therapeutically effective amount is within the ability of those skilled in the art in view of the present disclosure. In one embodiment, a therapeutically effective amount can be estimated using any appropriate animal model. The animal model is used to achieve a desired concentration range and an administration route. Thereafter, such information can be used to determine a dose and route useful for administration into humans. The exact dose of T regulatory cells is chosen by an individual physician in view of the condition of a patient to be treated. Doses and administration are adjusted to provide a sufficient level of the active portion, or to attain a desired effect. Preferably, a therapeutically effective amount of T regulatory cells will reduce a syndrome or a condition of discomfort of the subject associated with the spinal injury under treatment by at least about 20%, for example, by at least about 30%, about 40%, about 50%, about 60%, about 70%, about 80%, about 90%, or about 100%.

In a preferred embodiment of the present invention, the effective amount of T regulatory cells delivered to the area of spinal injury is about 10(4) to about 10(9) cells per administration.

To transplant T regulatory cells to the area of spinal injury, the T regulatory cells can be directly delivered to an exposed area of the spinal injury by means of injection. The T regulatory cells can also be delivered to the area of the spinal injury by means of a suitable vehicle. In a preferred embodiment of the present invention, the T regulatory cells are delivered to the desired area together with a fibrin glue. Fibrin glue has been used as an adhesive agent in various kinds of surgery, including neurosurgery. It is commercially available, from example, under the trademark Beriplast P™ (ZLB Behring, Germany). In a preferred embodiment, the fibrin glue used in embodiments of the present invention comprises fibrinogen, aprotinin and a calcium source that provides divalent calcium ions (such as calcium chloride or calcium carbonate).

Where fibrin glue is used, T regulatory cells can be delivered, before, simultaneously or after the delivery of a fibrin glue to an area of the spinal injury. In one embodiment, T regulatory cells can be mixed with fibrinogen and aprotinin before mixing with calcium chloride in the surgical area. For example, fibrinogen is first mixed with aprotinin, and further mixed with the calcium source in the surgical area to form a glue cast. In a fibrin glue used in the present invention, the concentration of fibrinogen can preferably be in the range of about 10 mg/ml to about 1000 mg/ml, and more preferably about 100 mg/ml; the concentration of aprotinin can preferably be in the range of about 10 KIU/ml to about 500 KIU/ml, more preferably about 200 KIU/ml; and the concentration of calcium chloride used as the calcium source can preferably be in the range of about 1 mM to about 100 mM, more preferably about 8 mM. In another embodiment of the present invention, transplantation of T regulatory cells to the area of spinal injury is achieved by using a hollow conduit filled with T regulatory cells to bridge the gap between severed ends of a transected spinal cord. The conduit used in the present invention is preferably composed of a biodegradable polymeric material conventionally used to make nerve conduits, including but not limited to, collagen, poly(lactic acid) (PLA), poly(glycolic acid) (PGA), poly(lactic-co-glycolic acid) (PLGA), polycaprolactone, poly(caprolactone-co-lactic acid) (PCLA), chitosan, alginate, hyaluronic acid, gelatin, and fibrin. The conduit used in the present invention can be any commercially available conduit composed of a biodegradable polymeric material. Alternatively, the hollow conduit can be fabricated as needed by any known method, such as the fiber templating process of Flynn et al. as set forth in Biomaterials 24: 4265-4272 (2003), and the low-pressure injection molding process of Sundback et al. as set forth in Biomaterials 24: 819-830 (2003); contents of both are incorporated herein by reference as if set out in full. In use, the severed ends of a transected spinal cord are brought into contact with the respective ends of a hollow conduit filled with T regulatory cells, which is slightly longer than the gap to be bridged, so that no tension is placed upon the severed spinal cord. Both the distal and proximal ends of the spinal cord are partially inserted into the conduit. If necessary, the severed ends can be sutured to the conduit over their perineurium. In another embodiment of the present invention, an effective amount of T regulatory cells are delivered to an area of a spinal injury in combination with one or more additional treatments for the spinal injury. For example, T regulatory cells can be used together with an effective dose of methylprednisolone, a treatment for acute traumatic spinal cord injuries. T regulatory cells can also be used together with chondroitinase treatment and other stem cell transplants. T regulatory cells can further be used in combination with Schwann cells bridge (SCs bridge), olfactory ensheathing glia and chondroitinase ABC. The effective amount of T regulatory cells can be combined with one or more additional treatments for the spinal injury in view of the present disclosure and the methods known to those skilled in the art.

In some embodiments T regulatory cells are administered at a time selected from the group consisting of: (a) greater than a week after the initial injury; (b) two weeks or greater after the initial injury; (c) three weeks or greater after the initial injury; (d) four weeks or greater after the initial injury; (e) two months or greater after the initial injury; (f) three months or greater after the initial injury; (g) four months or greater after the initial injury; (h) five months or greater after the initial injury; (i) six months or greater after the initial injury; (j) seven months or greater after the initial injury; (k) eight months or greater after the initial injury; (l) nine months or greater after the initial injury; (m) ten months or greater after the initial injury; (n) eleven months or greater after the initial injury; and (o) twelve months or greater after the initial injury. In one embodiment, the Nogo-receptor antagonist is administered three months or greater after the initial injury.

In some embodiment of the invention activity of T regulatory cells to accelerate healing and axonal reintegration is enhanced by administration of agents that inhibit NOGO and/or NOGO receptor. NOGA receptor antagonist polypeptides, aptamers and antibodies and fragments thereof are administered before, concurrent with or after T regulatory cell administration. For use in the methods disclosed herein can be composed of amino acids joined to each other by peptide bonds or modified peptide bonds, i.e., peptide isosteres, and may contain amino acids other than the 20 gene-encoded amino acids. NgR1 antagonist polypeptides, aptamers and antibodies and fragments thereof may be modified by natural processes, such as posttranslational processing, or by chemical modification techniques which are well known in the art. Such modifications are well described in basic texts and in more detailed monographs, as well as in a voluminous research literature. Modifications can occur anywhere in the NgR1 antagonist polypeptide, aptamer or antibody or fragments thereof, including the peptide backbone, the amino acid side-chains and the amino or carboxyl termini, or on moieties such as carbohydrates. It will be appreciated that the same type of modification may be present in the same or varying degrees at several sites in a given NgR1 antagonist polypeptide, aptamer or antibody or fragments thereof. Also, a given NgR1 antagonist polypeptide, aptamer or antibody or fragments thereof may contain many types of modifications. NgR1 antagonist polypeptides, aptamers or antibodies or fragments thereof may be branched, for example, as a result of ubiquitination, and they may be cyclic, with or without branching. Cyclic, branched, and branched cyclic NgR1 antagonist polypeptides, aptamers and antibodies or fragments thereof may result from posttranslational natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

For use in the treatment of spinal cord injury, in some embodiments of the invention, stimulation of T regulatory cells in vivo is accomplished by administration of Aldesleukin (Proleukin, Novartis), which is a commercially available IL-2 licensed for the treatment of metastatic renal cell carcinoma in the UK. It is produced by recombinant DNA technology using an Escherichia coli strain, which contains a genetically engineered modification of the human IL-2 gene, and is administered either intravenously or subcutaneously (SC). Following short intervenous infusion, its pharmacokinetic profile is typified by high plasma concentrations, rapid distribution into the extravascular space and a rapid renal clearance. The recommended doses for continuous infusion and subcutaneous injection (as detailed in the Summary of Product Characteristics) are repeated cycles of 18×106 IU per m2 per 24 hours for 5 days and repeated doses of 18×106 IU, respectively. Peak plasma levels are reached in 2-6 hours after SC administration, with bioavailability of aldesleukin ranging between 31% and 47%. The process of absorption and elimination of subcutaneous aldesleukin is described by a one-compartment model, with a 45 min absorption half-life and an elimination half-life of 3-5 hours.

In some embodiments of the invention, administration of angiogenic genes is performed in the spinal cord injury site to enhance efficacy of Treg cell therapy. Genes with angiogenic ability include: activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shingoingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1⊕1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF.

In one embodiment of the invention, patients suffering from spinal cord injury are pretreated with 0.3×106 IU of aldesleukin daily to stimulate production of endogenous T regulatory cells. In some embodiments of the invention, administration of low doses of IL-2 in the form of aldesleukin every day at concentrations of 0.3×106 to 3.0×106 IU IL-2 per square meter of body surface area for 8 weeks, or in other embodiments repetitive 5-day courses of 1.0×106 to 3.0×106 IU IL-2. Various types of IL-2 may be utilized. Examples of IL-2 variants, recombinant IL-2, methods of IL-2 production, methods of IL-2 purification, methods of formulation, and the like are well known in the art and can be found, for example, at least in U.S. Pat. Nos. 4,530,787, 4,569,790, 4,572,798, 4,604,377, 4,748,234, 4,853,332, 4,959,314, 5,464,939, 5,229,109, 7,514,073, and 7,569,215, each of which is herein incorporated by reference in their entirety for all purposes. In some embodiments, low dose interleukin-2 is provided together with activators of coinhibitory molecules, otherwise known as checkpoints. Such coinhibitory molecules include CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, A2aR, and combinations thereof. In some embodiments of the invention, mesenchymal stem cells are co-administered.

In other embodiments spinal cord injury patients are administered human IL-2 muteins that preferentially stimulate T regulatory (Treg) cells. These mutein proteins promote the proliferation, survival, activation and/or function of CD3+FoxP3+ T cells over CD3+FoxP3− T cells. Methods of measuring the ability to preferentially stimulate Tregs can be measured by flow cytometry of peripheral blood leukocytes, in which there is an observed increase in the percentage of FOXP3+CD4+ T cells among total CD4+ T cells, an increase in percentage of FOXP3+CD8+ T cells among total CD8+ T cells, an increase in percentage of FOXP3+ T cells relative to NK cells, and/or a greater increase in the expression level of CD25 on the surface of FOXP3+ T cells relative to the increase of CD25 expression on other T cells. Preferential growth of Treg cells can also be detected as increased representation of demethylated FOXP3 promoter DNA (i.e. the Treg-specific demethylated region, or TSDR) relative to demethylated CD3 genes in DNA extracted from whole blood, as detected by sequencing of polymerase chain reaction (PCR) products from bisulfite-treated genomic DNA. IL-2 muteins that preferentially stimulate Treg cells increase the ratio of CD3+FoxP3+ T cells over CD3+FoxP3− T cells in a subject or a peripheral blood sample at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000%.

In some embodiments of the invention, patients suffering from spinal cord injury are administered mesenchymal stem cells together with a tolerance inducing agent, said “agent” is meant to encompass essentially any type of molecule that can be used as a therapeutic properties to enhance T regulatory stimulating capable of mesenchymal stem cells administered in an allogeneic host. Proteins, such as antibodies, fusion proteins, and soluble ligands, any of which may either be identical to a wild-type protein or contain a mutation (i.e., a deletion, addition, or substitution of one or more amino acid residues), and the nucleic acid molecules that encode them (or that are “antisense” to them; e.g., an oligonucleotide that is antisense to the nucleic acids that encode a target polypeptide, or a component (e.g., a subunit) of their receptors), are all “agents.” The agents of the invention can either be administered systemically, locally, or by way of cell-based therapies (i.e., an agent of the invention can be administered to a patient by administering a cell that expresses that agent to the patient). A tolerance restoring agent can be .alpha.1-antitrypsin (AAT; sometimes abbreviated A1AT), which is also referred to as .alpha.1-proteinase inhibitor. AAT is a major serum serine-protease inhibitor that inhibits the enzymatic activity of numerous serine proteases including neutrophil elastase, cathespin G, proteinase 3, thrombin, trypsin and chymotrypsin. For example, one can administer an AAT polypeptide (e.g., a purified or recombinant AAT, such as human AAT) or a homolog, biologically active fragment, or other active mutant thereof. .alpha.1 proteinase inhibitors are commercially available for the treatment of AAT deficiencies, and include ARALAST™, PROLASTIN™. and ZEMAIRA™. The AAT polypeptide or the biologically active fragment or mutant thereof can be of human origin and can be purified from human tissue or plasma. Alternatively, it can be recombinantly produced. For ease of reading, we do not repeat the phrase “or a biologically active fragment or mutant thereof” after each reference to AAT. It is to be understood that, whenever a full-length, naturally occurring AAT can be used, a biologically active fragment or other biologically active mutant thereof (e.g., a mutant in which one or more amino acid residues have be substituted) can also be used. Similarly, we do not repeat on each occasion that a naturally occurring polypeptide (e.g., AAT) can be purified from a natural source or recombinantly produced. It is to be understood that both forms may be useful. Similarly, we do not repeatedly specify that the polypeptide can be of human or non-human origin. While there may be advantages to administering a human protein, the invention is not so limited.

The methods of the present invention (e.g., multiple-variable dose IL-2 alone or in combination with one or more other anti-immune disorder therapies) can be administered to a desired subject or once a subject is indicated as being a likely responder to such therapy. In another embodiment, the therapeutic methods of the present invention can be avoided if a subject is indicated as not being a likely responder to the therapy and an alternative treatment regimen, such as targeted and/or untargeted anti-immune therapies, can be administered.

In one embodiment, a multiple-variable IL-2 dose method of treating a subject suffering from spinal cord injury a therapy comprising a) administering to the subject an induction regimen comprising continuously administering to the subject interleukin-2 (IL-2) at a dose that increases the subject's plasma IL-2 level and increases the subject's ratio of immune suppressive T cells to conventional T lymphocytes (Tcons) and b) subsequently administering to the subject at least one maintenance regimen comprising continuously administering to the subject an IL-2 maintenance dose that is higher than the induction regimen dose and that i) further increases the subject's plasma IL-2 level and ii) further increases the ratio of immune suppressive T cells to Tcons, thereby treating the subject, is provided. In one embodiment, the level of plasma IL-2 resulting from the induction regimen is depleted below that of the prior peak plasma IL-2 level before the induction regimen. The IL-2 maintenance regimen can, in certain embodiments, increase the subject's plasma IL-2 level beyond the peak plasma IL-2 level induced by the induction regimen. The term “multiple-variable IL-2 dose method” refers to a therapeutic intervention comprising more than one IL-2 administration, wherein the more than one IL-2 administration uses more than one IL-2 dose. Such a method is contrasted from a “fixed” dosed method wherein a fixed amount of IL-2 is administered in a scheduled manner, such as daily. The term “induction regimen” refers to the continuous administration of IL-2 at a dose that increases the subject's plasma IL-2 level and increases the subject's immune suppressive T cells: Tcons ratio. In some embodiments, the regimen occurs until a peak level of plasma IL-2 is achieved. The subject's plasma IL-2 level and/or immune suppressive T cell: Tcons ratio can be increased by at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200% or more relative to the baseline ratio prior to initiation of therapy.

In one embodiment of the invention certain doses and methods according to FDA-approved uses, Tcons are preferentially activated relative to immune suppressive T cells such that the immune suppressive T cells: Tcons ratio actually decreases. By contrast, the methods of the present invention increase the immune suppressive T cells: Tcons ratio by using “low-dose IL-2” in a range determined herein to preferentially promote immune suppressive T cells over Tcons and that are safe and efficacious in subjects suffering from spinal cord injury.

The term “low-dose IL-2” refers to the dosage range wherein immune suppressive T cells are preferentially enhanced relative to Tcons. In one embodiment, low-dose IL-2 refers to IL-2 doses that are less than or equal to 50% of the “high-dose IL-2” doses (e.g., 18 million IU per m.sup.2 per day to 20 million IU per m.sup.2 per day, or more) used for anti-cancer immunotherapy. The upper limit of “low-dose IL-2” can further be limited by treatement adverse events, such as fever, chills, asthenia, and fatigue. IL-2 is generally dosed according to an amount measured in international units (IU) administered in comparison to body surface area (BSA) per given time unit. BSA can be calculated by direct measurement or by any number of well-known methods (e.g., the Dubois & Dubois formula), such as those described in the Examples. Generally, IL-2 is administered according in terms of IU per m.sup.2 of BSA per day. Exemplary low-dose IL-2 doses according to the methods of the present invention include, in terms of 10.sup.6 IU/m.sup.2/day, any one of 0.3, 0.4, 0.5, 0.6, 0.7, 0.8, 0.9, 1.0, 1.1, 1.2, 1.3, 1.4, 1.5, 1.6, 1.7, 1.8, 1.9, 2,0, 2.1, 2.2, 2.3, 2.4, 2.5, 2.6, 2.7, 2.8, 2.9, and 3.0.times.10.sup.6 IU/m.sup.2/day, including any values in between and/or ranges in between. For example, an induction regimen dose can range between 0.3.times.10.sup.6 IU/m.sup.2/day and 3.0.times.10.sup.6 IU/m.sup.2/day with any value or range in between. The term “continuous administration” refers to administration of IL-2 at regular intervals without any intermittent breaks in between. Thus, no interruptions in IL-2 occur. For example, the induction dose can be administered every day (e.g., once or more per day) during at least 1-14 consecutive days or any range in between (e.g., at least 4-7 consecutive days). As described herein, longer acting IL-2 agents and/or IL-2 agents administered by routes other than subcutaneous administration are contemplated. Intermittent intravenous administration of IL-2 described in the art results in short IL-2 half lives incompatible with increasing plasma IL-2 levels and increasing the immune suppressive T cells: Tcons ratio according to the present invention. However, once-daily subcutaneous IL-2 dosing, continuous IV infusion, long-acting subcutaneous IL-2 formulations, and the like are contemplated for achieving a persistent steady state IL-2 level.

As described above, IL-2 can be administered in a pharmaceutically acceptable formulation and by any suitable administration route, such as by subcutaneous, intravenous, intraperitoneal, oral, nasal, transdermal, or intramuscular administration. In one embodiment, the present invention provides pharmaceutically acceptable compositions which compose IL-2 at a therapeutically-effective amount, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate butler solutions; and (21) other non-toxic compatible substances employed in pharmaceutical formulations. Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent. In one embodiment, the Treg cell surface protein is selected from the group consisting of CD25, GITR, TIGIT, CTLA-4, neuropilin, OX40, LAG3, and combinations thereof, said Tregs are isolated possessing said surfaces proteins from a tissue source, and optionally expanded ex vivo prior to administration to a patient suffering from spinal cord injury.

In one embodiment, MSC exosomes, or particles may be produced by culturing mesenchymal stem cells in a medium to condition it. The mesenchymal stem cells may comprise human umbilical tissue derived cells which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes. Culture conditioned media may be concentrated by filtering/desalting means known in the art. In one embodiment Amicon filters, or substantially equivalent means, with specific molecular weight cut-offs are utilized, said cut-offs may select for molecular weights higher than 1 kDa to 50 kDa.

The cell culture supernatant may alternatively be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of stem cell or progenitor cell supernatant may be passed through each of these specific cartridges before elution, it is understood of one of skill in the art that larger cartridges may be used. After washing the cartridges material adsorbed is eluted with 3 ml methanol, evaporated under a stream of nitrogen, redissolved in a small volume of methanol, and stored at 4.degree. C.

Before testing the eluate for activity in vitro, the methanol is evaporated under nitrogen and replaced by culture medium. Said C18 cartridges are used to adsorb small hydrophobic molecules from the stem or progenitor cell culture supernatant, and allows for the elimination of salts and other polar contaminants. It may, however be desired to use other adsorption means in order to purify certain compounds from said fibroblast cell supernatant. Said fibroblast concentrated supernatant may be assessed directly for biological activities useful for the practice of this invention, or may be further purified. In one embodiment, said supernatant of fibroblast culture is assessed for ability to stimulate proteoglycan synthesis using an in vitro bioassay. Said in vitro bioassay allows for quantification and knowledge of which molecular weight fraction of supernatant possesses biological activity. Bioassays for testing ability to stimulate proteoglycan synthesis are known in the art. Production of various proteoglycans can be assessed by analysis of protein content using techniques including mass spectrometry, column chromatography, immune based assays such as enzyme linked immunosorbent assay (ELISA), immunohistochemistry, and flow cytometry.

Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Amniotic fluid stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

In one embodiment nucleus pulposus progenitors are administered, together with mesenchymal stem cell exosomes and/or mesenchymal stem cell conditioned media into the injured spine. In one embodiment nucleus pulposus progenitor cells are characterized as having high expression of CD47 (CD47.sup.hi) from the pluripotent stem cell population, thereby isolating one or more nucleus pulposus progenitor cells. In one embodiment, the method further comprises sorting the population for low CD26 expression (CD26.sup.lo), such that an isolated population of CD47.sup.hi/CD26.sup.lo nucleus pulposus progenitor cells is isolated. In another embodiment of this aspect and all other aspects described herein, the at least one differentiation-inducing agent comprises at least one of CHIR 99021, BMP4, KGF, FGF10, and retinoic acid. In one embodiment, the concentration of CHIR 99021 used with the methods of generating primordial nucleus pulposus progenitors as described herein comprises at least 0.5 .mu.M, at least 1 .mu.M, at least 1.5 .mu.M, at least 2 .mu.M, at least 2.5 .mu.M, at least 3 .mu.M, at least 3.5 .mu.M, at least 4 .mu.M, at least 4.5 .mu.M, at least 5 .mu.M, at least 1004, at least 20 .mu.M or more. In another embodiment, the concentration of CHIR 99021 used with the methods of generating primordial nucleus pulposus progenitors as described herein comprises a concentration in the range of 1-5 .mu.M, 1-10 .mu.M, 1-20 .mu.M, 2-4 .mu.M, 5-20 .mu.M, 10-20 .mu.M, or any range there between. In another embodiment, the concentration of BMP4 used with the methods of generating primordial nucleus pulposus progenitors as described herein comprises at least 1 ng/ml, at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 20 ng/ml, at least 30 ng/ml, at least 40 ng/ml, at least 50 ng/ml, at least 60 ng/ml, at least 75 ng/ml, at least 100 ng/ml, at least 125 ng/ml, at least 150 ng/ml, at least 200 ng/ml or more. In another embodiment, the concentration of BMP4 used with the methods of generating primordial nucleus pulposus progenitors as described herein comprises a concentration in the range of 1-50 ng/ml, 1-25 ng/ml, 1-10 ng/ml, 5-10 ng/ml, 5-15 ng/ml, 5-25 ng/ml, 25-50 ng/ml, 25-75 ng/ml, 25-100 ng/mL, 25-150 ng/mL, 75-125 ng/ml or any range therebetween.

1. A method of treating spinal cord injury comprising administration of a population of cells in which one cell type of said populations is a T cell possessing ability to inhibit proliferation of other T cells.

2. The method of claim 1, wherein said T cell capable of suppressing proliferation of other T cells is capable of enhancing production of TGF-beta from said other T cells.

3. The method of claim 1, wherein said T cell capable of suppressing proliferation of other T cells is capable of enhancing production of HLA-G from said other T cells.

4. The method of claim 1, wherein said T cell capable of suppressing proliferation of other T cells is a T regulatory cell.

5. The method of claim 4, wherein said T regulatory cell is generated from perinatal tissues.

6. The method of claim 5, wherein said T regulatory cells are isolated by cutting perinatal tissue into pieces approximately 1-5 millimeters, digesting said tissue with a mixture of dissociative enzymes, filtering said cells to remove tissue aggregates and culturing in a liquid media.

7. The method of claim 6, wherein said liquid media is AIM-V media.

8. The method of claim 7, wherein said liquid media is supplemented with mitogens selective for T regulatory cells.

9. The method of claim 8, wherein said mitogen selective for T regulatory cells is interleukin-2 together with TGF-beta.

10. The method of claim 4, wherein said T regulatory cell is isolated from peripheral blood.

11. The method of claim 4, wherein said T regulatory cells are generated from pluripotent stem cells.

12. The method of claim 11, wherein said pluripotent stem cells are generated from induced pluripotent stem cells.

13. A method of treating spinal cord injury comprising administration of T regulatory cells in the area of injury using a localization means.

14. The method of claim 13, wherein said localization means is a decellularized tissue scaffold.

15. The method of claim 14, wherein said decellularized tissue scaffold is derived from perinatal tissue.

16. The method of claim 14, wherein said decellularized tissue scaffold is derived from cadaveric spinal cord tissue.

17. The method of claim 13, wherein a mesenchymal stem cell population is administered to the area of injury.

18. The method of claim 17, wherein said mesenchymal stem cell is transfected with a growth factor.

19. The method of claim 18, wherein said growth factor is FGF-1.

20. The method of claim 19, wherein said growth factor is TGF-beta.
